(19) United States
(12) Patent Application Publication
(10) Pub. No.: US 2005/0262588 A1
(43) Pub. Date: Nov. 24, 2005

(54) Thioesterase-related nucleic acid sequences and methods of use for the production of plants with modified fatty acid composition

(76) Inventors: Katayoon Dehesh, Vacaville, CA (US); Vic C. Knauf, Bainbridge Island, WA (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 10/518,752

(22) PCT Filed: Jun. 20, 2003
(86) PCT No.: PCT/US03/19441

§ 371 Date: Jul. 1, 2005

Related U.S. Application Data
(60) Provisional application No. 60/390,185, filed on Jun. 21, 2002.

(51) Int. Cl. A01H 1/00; C12N 15/82; A01H 5/00; C12N 5/04; C12N 9/04

(52) U.S. Cl. 800/281; 800/312; 435/190; 435/415; 435/468

(57) ABSTRACT

The present invention is directed to nucleic acid molecules and nucleic acid constructs, and other agents associated with fatty acid synthesis, particularly the ratios of saturated and unsaturated fats. Moreover, the present invention is directed to plants incorporating such agents where the plants exhibit altered ratios of saturated and unsaturated fats. In particular, the present invention is directed to plants incorporating such agents where the plants exhibit altered levels of saturated and unsaturated fatty acids.

### FIELD OF THE INVENTION

The present invention is directed to nucleic acid molecules and nucleic acid constructs, and other agents associated with fatty acid synthesis. Moreover, the present invention is directed to plants incorporating such agents where the plants exhibit altered ratios of saturated and unsaturated fats. In particular, the present invention is directed to plants incorporating such agents where the plants exhibit altered ratios of saturated to unsaturated fatty acids.

### BACKGROUND

Plant oils are used in a variety of applications. Novel vegetable oil compositions and improved approaches to obtain oil compositions, from biosynthetic or natural plant sources, are needed. Depending upon the intended oil use, various different fatty acid compositions are desired. Plants, especially plant species which synthesize large amounts of oils in plant seeds, are an important source of oils both for edible and industrial uses.

With the exception of coconut endosperm and palm kernel oils, which contain high amounts of laurate (C12:0), the common edible oils all basically consist of palmitate (16:0), stearate (18:0), oleate (18:1), linoleate (18:2), and linolenate (18:3). Many oilseed species have highly elevated levels of saturated fatty acids. Coconut oil contains more than 90% saturated fatty acids, predominantly laurate (12:0) and other medium chain fatty acids ranging from C6 to C16. Other highly saturated oils include oils with high palmitate (16:0) and stearate (18:0) levels (up to approximately 60% of acyl chains). These oils include those derived from cocoa butter (25% palmitate; 34% stearate) and oil palm mesocarp (45% palmitate; 15% stearate). Soybean oil typically contains about 16-20% saturated fatty acids: 13-16% palmitate and 3-4% stearate. Voelker et al., 52 Annu. Rev. Plant Physiol. Plant Mol. Biol. 335-61 (2001).

For many oil applications, saturated fatty acid levels are preferably less than 6% by weight, and more preferably about 2-3% by weight. Saturated fatty acids have undesirable high melting points and cloud at low temperatures. Products created from oils containing low saturated fatty acid levels may be preferred by consumers and the food industry because they are perceived to be healthier and/or may be labeled as “saturated fat free” or “trans fat free” products in accordance with FDA guidelines. Oils with low saturated fatty acid levels have improved cold flow properties, which are important in biodiesel and lubricant applications, and do not cloud at low temperatures, thereby reducing or eliminating the need to winterize the oil for food applications such as salad oils.

Higher plants synthesize fatty acids in the plastids via the fatty acid synthetase (FAS) pathway. In developing oilseeds, most fatty acids are attached to glycerol backbones to form triglycerides, for storage as a source of energy.

β-ketoacyl-ACP synthase I catalyzes elongation up to palmitoyl-ACP (C 16:0), whereas β-ketoacyl-ACP synthase II catalyzes the final elongation to stearoyl-ACP (C18:0). Common plant unsaturated fatty acids, such as oleic, linoleic and linolenic acids found in storage triglycerides, originate from the desaturation of stearoyl-ACP to form oleoyl-ACP (C18: 1) in a reaction catalyzed by a soluble plastid delta-9 desaturase (also often referred to as “stearoyl-ACP desaturase”). Additional desaturation is effected sequentially by the actions of membrane bound delta-12 desaturase and delta-15 desaturase. These “desaturases” thus create polyunsaturated fatty acids.

Specific thioesterases can terminate fatty acid chain elongation by hydrolyzing the newly produced acyl-ACPs into free fatty acids and ACP. Subsequently, the free fatty acids are converted to fatty acyl-CoAs in the plastid envelope and exported to the cytoplasm, where they may be incorporated into the endoplasmic reticulum (ER) lipid biosynthesis pathway (Kennedy pathway), which is responsible for the formation of phospholipids, triglycerides, and other neutral lipids. Plant acyl-ACP thioesterases are of biochemical interest because of their roles in fatty acid synthesis and their utility in bioengineering of plant oil seeds. The thioesterases have an important role in determining chain length during de novo fatty acid biosynthesis in plants, and thus these enzymes are useful in the provision of various modifications of fatty acyl compositions, particular with respect to the relative proportions of various fatty acyl groups that are present in seed storage oils.

Plant thioesterases can be classified into two gene families based on sequence homology and substrate preference. The first gene family, FATA, includes long chain acyl-ACP thioesterases having activity primarily on oleoyl-ACP (18:1-ACP). Oleoyl-ACP is the immediate precursor of most fatty acids found in phospholipids and triglycerides synthesized by the ER lipid biosynthetic pathway. A second class of plant thioesterases, FATB, includes enzymes that, in most plants, utilize palmitoyl-ACP (16:0-ACP), stearoyl (18:0-ACP), and oleoyl-ACP (18:1-ACP). FATB enzymes have been isolated from California bay (Umbellularia californica) (U.S. Pat. No. 5,955,329; U.S. Pat. No. 5,723,761), elm (U.S. Pat. No. 5,723,761), Cuphea hookeriana (U.S. Pat. No. 5,723,761), Cuphea palustris (U.S. Pat. No. 5,955,329), Cuphea lanceolata, nutmeg, Arabidopsis thaliana, mango (U.S. Pat. No. 5,723,761), leek (U.S. Pat. No. 5,723,761), camphor (Cinnamomum camphora) (U.S. Pat. No. 5,955,329), canola (U.S. Pat. No. 5,955,650), and maize (U.S. Pat. No. 6,331,664).

Obtaining nucleic acid sequences capable of producing a phenotypic result in FAS, desaturation and/or incorporation of fatty acids into a glycerol backbone to produce an oil is subject to various obstacles including but not limited to the identification of metabolic factors of interest, choice and characterization of an enzyme source with useful kinetic properties, purification of the protein of interest to a level which will allow for its amino acid sequencing, utilizing amino acid sequence data to obtain a nucleic acid sequence capable of use as a probe to retrieve the desired DNA sequence, and the preparation of gene constructs, transformation and analysis of the resulting plants.

Thus, additional nucleic acid targets and methods for modifying fatty acid compositions are needed. In particular, constructs and methods to produce a variety of ranges of different fatty acid compositions are needed.

### SUMMARY OF THE INVENTION

The present invention provides a substantially purified nucleic acid molecule comprising a nucleic acid sequence with at least 70% sequence identity to SEQ ID NO: 2 or its complement. Also provided by the present invention is a substantially purified nucleic acid molecule comprising a nucleic acid sequence with at least 70% sequence identity to SEQ ID NO: 3 or its complement. Also provided by the present invention is a substantially purified nucleic acid molecule comprising a nucleic acid sequence with at least 70% sequence identity to SEQ ID NO: 4 or its complement. Also provided by the present invention is a substantially purified nucleic acid molecule comprising a nucleic acid sequence with at least 70% sequence identity to SEQ ID NO: 5 or its complement. Also provided by the present invention is a substantially purified nucleic acid molecule comprising a nucleic acid sequence with at least 70% sequence identity to SEQ ID NO: 6 or its complement. Also provided by the present invention is a substantially purified nucleic acid molecule comprising a nucleic acid sequence with at least 70% sequence identity to SEQ ID NO: 7 or its complement. Also provided by the present invention is a substantially purified nucleic acid molecule comprising a nucleic acid sequence with at least 70% sequence identity to SEQ ID NO: 8 or its complement. Further provided by the present invention are a nucleic acid molecule comprising at least 15 consecutive nucleotides of a nucleic acid molecule having the sequence of SEQ ID NO: 2; and a nucleic acid molecule comprising at least 15 consecutive nucleotides of a nucleic acid molecule having the sequence of SEQ ID NO: 3; and a nucleic acid molecule comprising at least 15 consecutive nucleotides of a nucleic acid molecule having the sequence of SEQ ID NO: 4; and a nucleic acid molecule comprising at least 15 consecutive nucleotides of a nucleic acid molecule having the sequence of SEQ ID NO: 5; and a nucleic acid molecule comprising at least 15 consecutive nucleotides of a nucleic acid molecule having the sequence of SEQ ID NO: 6; and a nucleic acid molecule comprising at least 15 consecutive nucleotides of a nucleic acid molecule having the sequcnce of SEQ ID NO: 7; and a nucleic acid molecule comprising at least 15 consecutive nucleotides of a nucleic acid molecule having the sequence of SEQ ID NO: 8.

Also provided by the present invention is a recombinant nucleic acid molecule comprising as operably linked components: (A) a promoter that functions in a plant cell to cause production of an mRNA molecule; and (B) a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either.

Also provided by the present invention is an intron obtained from a genomic polynucleotide sequence wherein the genomic polynucleotide sequence is selected from the group consisting of: (a) a genomic polynucleotide sequence having at least 70% identity to coding regions of SEQ ID NO: 1 over the entire length of SEQ ID NO: 1; (b) a genomic polynucleotide sequence having at least 80% identity to coding regions of SEQ ID NO: 1 over the entire length of SEQ ID NO: 1; (c) a genomic polynucleotide sequence having at least 90% identity to coding regions of SEQ ID NO: 1 over the entire length of SEQ ID NO: 1; and (d) a genomic polynucleotide sequence having at least 95% identity to coding regions of SEQ ID NO: 1 over the entire length of SEQ ID NO: 1.

Also provided by the present invention is an intron obtained from a genomic polynucleotide sequence wherein the genomic polynucleotide sequence is selected from the group consisting of: (a) a genomic polynucleotide sequence having at least 70% identity to coding regions of SEQ ID NO: 10 over the entire length of SEQ ID NO: 10; (b) a genomic polynucleotide sequence having at least 80% identity to coding regions of SEQ ID NO: 10 over the entire length of SEQ ID NO: 10; (c) a genomic polynucleotide sequence having at least 90% identity to coding regions of SEQ ID NO: 10 over the entire length of SEQ ID NO: 10; and (d) a genomic polynucleotide sequence having at least 95% identity to coding regions of SEQ ID NO: 10 over the entire length of SEQ ID NO: 10.

Also provided by the present invention are transformed plant cells and plants comprising a recombinant nucleic acid molecule, the recombinant nucleic acid molecule comprising as operably linked components: (A) a promoter that functions in a plant cell to cause production of an mRNA molecule; and (B) a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either.

The present invention also provides a transformed soybean plant having a recombinant nucleic acid molecule that comprises a promoter operably linked to a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either.

Further provided by the present invention is a transformed soybean plant having a nucleic acid molecule that comprises (a) a first promoter operably linked to a first nucleic acid molecule having a first nucleic acid sequence that has 85% or greater identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, and (b) a second nucleic acid molecule having a second nucleic acid sequence that encodes an enzyme selected from the group consisting of beta-ketoacyl-ACP synthase 1, beta-ketoacyl-ACP synthase IV, and delta-9 desaturase.

The present invention also provides seed derived from a transformed plant which comprises a recombinant nucleic acid molecule, the recombinant nucleic acid molecule comprising as operably linked components: (A) a promoter that functions in a plant to cause production of an mRNA molecule; and (B) a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either.

Also provided by the present invention is oil derived from seed of a transformed plant, where the transformed plant comprises a recombinant nucleic acid molecule which comprises as operably linked components: (A) a promoter that functions in a plant to cause production of an mRNA molecule; and (B) a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, where the oil exhibits a reduced saturated fatty acid content relative to oil derived from seed of a plant with a similar genetic background but lacking the recombinant nucleic acid molecule.

The present invention also provides a method of producing a transformed plant having seed with a reduced saturated fatty acid content comprising: (A) transforming a plant with a nucleic acid molecule to produce a transformed plant, where the nucleic acid molecule comprises a nucleic acid sequence that has 85% or greater identity to an intron of a plant thioesterase gene; and (B) growing the transformed plant, where the plant produces seed with a reduced saturated fatty acid content relative to a plant having a similar genetic background but lacking the nucleic acid molecule.

Further provided by the present invention is a method of producing a plant having a seed with reduced palmitic and stearic acid levels comprising: transforming a plant with a nucleic acid molecule that comprises (a) a first promoter operably linked to a first nucleic acid molecule having a first nucleic acid sequence that has 85% or greater identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, and (b) a second nucleic acid molecule having a second nucleic acid sequence that encodes an enzyme selected from the group consisting of beta-ketoacyl-ACP synthase I, beta-ketoacyl-ACP synthase IV, and delta-9 desaturase; and growing the plant, where the plant produces seed with reduced palmitic and stearic acid levels relative to a plant having a similar genetic background but lacking the nucleic acid molecule.

The present invention also provides a method of producing a plant having a seed with a modified oil composition comprising: transforming a plant with a nucleic acid molecule that comprises, as operably linked components, a first promoter and a first nucleic acid molecule having a first nucleic acid sequence that has 85% or greater identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either; and, growing the plant, where the plant produces seed with a modified oil composition relative to a plant having a similar genetic background but lacking the nucleic acid molecule.

The present invention further provides a method of modifying the lipid composition in a plant cell comprising: transforming a plant cell with a recombinant DNA construct having a DNA sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, and growing the cell under conditions where transcription of the DNA sequence is initiated, where the lipid composition is modified.

Also provided by the present invention is a method of modifying the lipid composition in a host cell comprising: transforming a host cell with a DNA construct comprising as operably associated components in the 5′ to 3′ direction of transcription, a transcriptional initiation region functional in the host cell, a DNA sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, and a transcription termination sequence, and growing the cell under conditions where transcription of the DNA sequence is initiated, where the lipid composition is modified.

Further provided by the present invention is a method of altering the expression of a FATB gene in a seed comprising: (a) introducing into a plant cell a first DNA sequence capable of expressing a first RNA that exhibits at least 90% identity to a transcribed intron of the FATB gene, and a second DNA sequence capable of expressing a second RNA capable of forming a dsRNA with the first RNA; and (b) expressing the first RNA and the second RNA in a seed.

Also provided by the present invention are methods of altering the expression of a FATB gene in a seed comprising: (a) introducing into a plant cell a first DNA sequence capable of expressing an RNA that exhibits at least 90% identity to a transcribed intron of the FATB gene and a second DNA sequence capable of expressing a second RNA that exhibits at least 90% identity to a transcribed intron of the FATB gene; and (b) expressing said the first RNA and the second RNA in a seed.

### BRIEF DESCRIPTION OF THE DRAWINGS

FIG. 1 is a schematic of construct pCGN3892.

FIG. 2 is a schematic of construct pMON70674.

FIG. 3 is a schematic of construct pMON41164.

FIG. 4 is a schematic of construct pMON70678.

FIG. 5 is a schematic of construct pMON70675.

FIG. 6 is a schematic of construct pMON70680.

FIG. 7 is a schematic of construct pMON70656.

FIG. 8 is a schematic of construct pMON70681.

### DETAILED DESCRIPTION OF THE INVENTION

Description of the Nucleic Acid Sequences

SEQ ID NO: 1 sets forth a nucleic acid sequence of a soybean FATB genomic clone.

SEQ ID NO: 2 sets forth a nucleic acid sequence of a soybean FATB intron I.

SEQ ID NO: 3 sets forth a nucleic acid sequence of a soybean FATB intron II.

SEQ ID NO: 4 sets forth a nucleic acid sequence of a soybean FATB intron III.

SEQ ID NO: 5 sets forth a nucleic acid sequence of a soybean FATB intron IV.

SEQ ID NO: 6 sets forth a nucleic acid sequence of a soybean FATB intron V.

SEQ ID NO: 7 sets forth a nucleic acid sequence of a soybean FATB intron VI.

SEQ ID NO: 8 sets forth a nucleic acid sequence of a soybean FATB intron VII.

SEQ ID NO: 9 sets forth an amino acid sequence of a soybean FATB enzyme.

SEQ ID NO: 10 sets forth a nucleic acid sequence of a soybean FATB partial genomic clone.

SEQ ID NOs: 11-18 set forth nucleic acid sequences of oligonucleotide primers.

SEQ ID NO: 19 sets forth a nucleic acid sequence of a PCR product containing soybean FATB intron II.

SEQ ID NO: 20 sets forth a nucleic acid sequence of a soybean FATB cDNA.

Definitions

As used herein, the term “gene” is used to refer to the nucleic acid sequence that encompasses the 5′ promoter region associated with the expression of the gene product, any intron and exon regions and 3′ untranslated regions associated with the expression of the gene product.

As used herein, the term “ACP” is used to refer to an acyl carrier protein moiety. The term “fatty acid”, as used herein, refers to free fatty acids and acyl-fatty acid groups.

As used herein, a “FATB” or “palmitoyl-ACP thioesterase” gene is a gene that encodes an enzyme (FATB) capable of catalyzing the hydrolytic cleavage of the carbon-sulfur thioester bond in the panthothene prosthetic group of palmitoyl-ACP as its preferred reaction. Hydrolysis of other fatty acid-ACP thioesters may also be catalyzed by this enzyme.

When referring to proteins and nucleic acids herein, the use of plain capitals, e.g., “FATB”, indicates a reference to an enzyme, protein, polypeptide, or peptide, and the use of italicized capitals, e.g., “FATB”, is used to refer to nucleic acids, including without limitation genes, cDNAs, and mRNAs.

As used herein, a “beta-ketoacyl-ACP synthase I” or “KAS I” gene is a gene that encodes an enzyme (KAS I) capable of catalyzing the elongation of a fatty acyl moiety up to palmitoyl-ACP (C16:0). Exemplary KAS I genes and enzymes are described in U.S. Pat. No. 5,475,099 and PCT Publication WO 94/10189.

As used herein, a “beta-ketoacyl-ACP synthase IV” or “KAS IV” gene is a gene that encodes an enzyme (KAS IV) capable of catalyzing the condensation of medium chain acyl-ACPs. Exemplary KAS IV genes and enzymes are described in PCT Publication WO 98/46776.

As used herein, a “delta-9 desaturase” or “stearoyl-ACP desaturase” or “omega-9 desaturase” gene is a gene that encodes an enzyme capable of catalyzing the insertion of a double bond into a fatty acyl moiety at the ninth position counted from the carboxyl terminus. Exemplary delta-9 desaturase genes and enzymes are described in U.S. Pat. No. 5,723,595.

As used herein, a “mid-oleic soybean seed” is a seed having between 50% and 75% oleic acid present in the oil composition of the seed.

As used herein, a “high oleic soybean seed” is a seed with oil having greater than 75% oleic acid present in the oil composition of the seed.

As used herein, a “low saturate” oil composition contains between 3.4 and 7 percent saturated fatty acids.

As used herein, a “zero saturate” oil composition contains less than 3.4 percent saturated fatty acids.

As used herein, a cell or organism can have a family of more than one gene encoding a particular enzyme, e.g., a plant can have a family of more than one FATB gene (i.e., genes which encode an enzyme with the specified activity present at different locations within the genome of the plants). As used herein, a “FATB gene family member” is any FATB gene found within the genetic material of the plant. In one embodiment, a gene family can be additionally classified by the similarity of the nucleic acid sequences. In a preferred aspect of this embodiment, a gene family member exhibits at least 60%, more preferably at least 70%, more preferably at least 80% nucleic acid sequence identity in the coding sequence portion of the gene.

The term “non-coding” refers to sequences of nucleic acid molecules that do not encode part or all of an expressed protein. Non-coding sequences include but are not limited to introns, promoter regions, 3′ untranslated regions, and 5′ untranslated regions.

The term “intron” as used herein refers to the normal sense of the term as meaning a segment of nucleic acid molecules, usually DNA, that does not encode part of or all of an expressed protein, and which, in endogenous conditions, is transcribed into RNA molecules, but which is spliced out of the endogenous RNA before the RNA is translated into a protein.

The term “exon” as used herein refers to the normal sense of the term as meaning a segment of nucleic acid molecules, usually DNA, that encodes part of or all of an expressed protein.

As used herein, a promoter that is “operably linked” to one or more nucleic acid sequences is capable of driving expression of one or more nucleic acid sequences, including multiple coding or non-coding nucleic acid sequences arranged in a polycistronic configuration.

A “polycistronic gene” or “polycistronic mRNA” is any gene or mRNA that contains transcribed nucleic acid sequences which correspond to nucleic acid sequences of more than one gene targeted for expression. It is understood that such polycistronic genes or mRNAs may contain sequences that correspond to introns, 5′UTRs, 3′UTRs, or combinations thereof, and that a recombinant polycistronic gene or mRNA might, for example without limitation, contain sequences that correspond to one or more UTRs from one gene and one or more introns from a second gene.

As used herein, the term complement of a nucleic acid sequence refers to the complement of the sequence along its complete length.

As used herein, any range set forth is inclusive of the end points of the range unless otherwise stated.

Agents

The agents of the invention will preferably be “biologically active” with respect to either a structural attribute, such as the capacity of a nucleic acid molecule to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response. The agents will preferably be “substantially purified.” The term “substantially purified,” as used herein, refers to a molecule separated from substantially all other molecules normally associated with it in its native environmental conditions. More preferably a substantially purified molecule is the predominant species present in a preparation. A substantially purified molecule may be greater than 60% free, greater than 75% free, preferably greater than 90% free, and most preferably greater than 95% free from the other molecules (exclusive of solvent) present in the natural mixture. The term “substantially purified” is not intended to encompass molecules present in their native environmental conditions.

The agents of the invention may also be recombinant. As used herein, the term “recombinant” means any agent (e.g., including but limited to DNA, peptide), that is, or results, however indirectly, from human manipulation of a nucleic acid molecule.

It is understood that the agents of the invention may be labeled with reagents that facilitate detection of the agent (e.g., fluorescent labels, Prober et al., Science 238:336-340 (1987); Albarella et al., EP 144914; chemical labels, Sheldon et al., U.S. Pat. No. 4,582,789; Albarella et al., U.S. Pat. No. 4,563,417; modified bases, Miyoshi et al., EP 119448).

Nucleic Acid Molecules

Agents of the invention include nucleic acid molecules. In an aspect of the present invention, the nucleic acid molecule comprises a nucleic acid sequence, which when introduced into a cell or organism, is capable of selectively reducing the level of a FATB protein and/or FATB transcript.

In a preferred aspect of the invention, the nucleic acid sequences are intron sequences or other non-coding sequences of a FATB gene, which when introduced into a cell or organism are capable of selectively reducing the level of an endogenous FATB protein and/or endogenous FATB transcript, thereby resulting in a modification of the fatty acid biosynthetic pathway and a consequent decrease in the levels of saturated fatty acids in the cell or organism. Non-coding sequences of a FATB gene may also be used in combination with nucleic acid sequences coding for enzymes such as beta-ketoacyl-ACP synthase I, beta-ketoacyl-ACP synthase IV, and delta-9 desaturase, which further modifies the fatty acid biosynthetic pathway and further decreases the level of saturated fatty acids in the cell or organism. Non-coding sequences of a FATB gene may also be used in combination with nucleic acid sequences that down-regulate other enzymes, for example a CDNA that is capable of sense suppression of a delta-12 desaturase gene, which further modifies the fatty acid biosynthetic pathway and further decreases the level of saturated fatty acids in the cell or organism.

In a preferred aspect, the capability of a nucleic acid molecule to selectively reduce the level of a protein and/or transcript is carried out by a comparison of levels of mRNA transcripts. In another preferred aspect of the present invention, the nucleic acid molecule of the invention comprises a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7SEQ ID NO: 8, complements thereof, and fragments of either.

In one aspect of the present invention the nucleic acids of the present invention are said to be introduced nucleic acid molecules. A nucleic acid molecule is said to be “introduced” if it is inserted into a cell or organism as a result of human manipulation, no matter how indirect. Examples of introduced nucleic acid molecules include, but are not limited to, nucleic acids that have been introduced into cells via transformation, transfection, injection, and projection, and those that have been introduced into an organism via methods including, but not limited to, conjugation, endocytosis, and phagocytosis. The cell or organism can be, or can be derived from, without limitation, a plant, plant cell, algae cell, algae, fungal cell, fungus, or bacterial cell.

As used herein, “a selective reduction” of an agent such as a protein, fatty acid, or mRNA is relative to a cell or organism lacking a nucleic acid molecule capable of selectively reducing the agent. In a preferred aspect, the level of the agent is selectively reduced by at least 50%, preferably at least more than 75%, and even more preferably by at least more than 30 90% or 95%.

As used herein, “a partial reduction” of the level of an agent such as a protein, fatty acid, or mRNA means that the level is reduced at least 25% relative to a cell or organism lacking a nucleic acid molecule capable of reducing the agent.

As used herein, “a substantial reduction” of the level of an agent such as a protein, fatty acid, or mRNA means that the level is reduced at least 75% relative to a cell or organism lacking a nucleic acid molecule capable of reducing the agent.

As used herein, “an effective elimination” of an agent such as a protein, fatty acid, or mRNA means that the level of the agent is reduced at least 95% relative to a cell or organism lacking a nucleic acid molecule capable of reducing the agent.

When levels of an agent are compared, such a comparison is preferably carried out between organisms with a similar genetic background. In a preferred aspect, a similar genetic background is a background where the organisms being compared share 50% or greater of their nuclear genetic material. In a more preferred aspect a similar genetic background is a background where the organisms being compared share 75% or greater, even more preferably 90% or greater of their nuclear genetic material. In another even more preferable aspect, a similar genetic background is a background where the organisms being compared are plants, and the plants are isogenic except for any genetic material originally introduced using plant transformation techniques.

In an embodiment of the present invention, a nucleic acid molecule, when introduced into a cell or organism, is capable of selectively reducing the level of a protein, fatty acid, and/or transcript. In a preferred aspect, the capability of a nucleic acid molecule to selectively reduce the level of a protein, fatty acid, and/or transcript is determined relative to a cell or organism lacking a nucleic acid molecule capable of selectively reducing the protein, fatty acid, and/or transcript. As used herein, mRNA transcripts include processed and non-processed mRNA transcripts, and a “FATB transcript” refers to any transcript encoded by a FATB gene.

In another embodiment, a nucleic acid molecule, when introduced into a cell or organism, is capable of at least partially reducing the level of a FATB protein and/or FATB transcript. In a different embodiment, a nucleic acid molecule, when introduced into a cell or organism, is capable of at least substantially reducing the level of a FATB protein and/or FATB transcript. In a further embodiment, a nucleic acid molecule, when introduced into a cell or organism, is capable of effectively eliminating the level of a FATB protein and/or FATB transcript.

In a different embodiment, a nucleic acid molecule, when introduced into a cell or organism, is capable of selectively reducing the level of a FATB protein and/or FATB transcript while overexpressing the level of a different protein and/or transcript. Preferably, the different protein is selected from the group consisting of beta-ketoacyl-ACP synthase I, beta-ketoacyl-ACP synthase IV, and delta-9 desaturase, and the different transcript encodes an enzyme selected from the group consisting of beta-ketoacyl-ACP synthase I, beta-ketoacyl-ACP synthase IV, and delta-9 desaturase.

In a further embodiment, a nucleic acid molecule, when introduced into a cell or organism, is capable of at least partially reducing the level of a FATB protein and/or FATB transcript while overexpressing the level of a different protein and/or transcript. Preferably, the different protein is selected from the group consisting of beta-ketoacyl-ACP synthase I, beta-ketoacyl-ACP synthase IV, and delta-9 desaturase, and the different transcript encodes an enzyme selected from the group consisting of beta-ketoacyl-ACP synthase I, beta-ketoacyl-ACP synthase IV, and delta-9 desaturase. In a different embodiment, a nucleic acid molecule, when introduced into a cell or organism, is capable of at least substantially reducing the level of a FATB protein and/or FATB transcript while overexpressing the level of a different protein and/or transcript. In a further embodiment, a nucleic acid molecule, when introduced into a cell or organism, is capable of effectively eliminating the level of a FATB protein and/or FATB transcript while overexpressing the level of a different protein and/or transcript.

Further preferred embodiments of the invention are nucleic acid molecules that are at least 50%, 60%, or 70% identical over their entire length to a nucleic acid molecule of the invention, and nucleic acid molecules that are complementary to such nucleic acid molecules. More preferable are nucleic acid molecules that comprise a region that is at least 80% or 85% identical over its entire length to a nucleic acid molecule of the invention and nucleic acid molecules that are complementary thereto. In this regard, nucleic acid molecules at least 90% identical over their entire length are particularly preferred, those at least 95% identical are especially preferred. Further, those with at least 97% identity are highly preferred and those with at least 98% and 99% identity are particularly highly preferred, with those at least 99% being the most highly preferred.

The invention also provides a nucleic acid molecule comprising a nucleic acid molecule sequence obtainable by screening an appropriate library containing the complete gene for a nucleic acid molecule sequence set forth in the Sequence Listing under stringent hybridization conditions with a probe having the sequence of said nucleic acid molecule sequence or a fragment thereof; and isolating said nucleic acid molecule sequence. Fragments useful for obtaining such a nucleic acid molecule include, for example, probes and primers as described herein.

Nucleic acid molecules of the invention can be used as a hybridization probe for RNA, cDNA, or genomic DNA to isolate full length cDNAs or genomic clones and to isolate cDNA or genomic clones of other genes that have a high sequence similarity to a nucleic acid molecule set forth in the Sequence Listing.

The nucleic acid molecules of the present invention can be readily obtained by using the herein described nucleic acid molecules or fragments thereof to screen cDNA or genomic libraries obtained from plant species or other appropriate organisms. These methods are known to those of skill in the art, as are methods for forming such libraries. In one embodiment, such sequences are obtained by incubating nucleic acid molecules of the present invention with members of genomic libraries and recovering clones that hybridize to such nucleic acid molecules thereof. In a second embodiment, methods of chromosome walking or inverse PCR may be used to obtain such sequences. In a third embodiment, the sequence of a nucleic acid molecule of the present invention may be used to screen a library or database, using bioinformatics techniques known in the art. See, e.g., Bioinformatics, Baxevanis & Ouellette, eds., Wiley-Interscience (1998).

Any of a variety of methods may be used to obtain one or more of the nucleic acid molecules of the present invention. Automated nucleic acid synthesizers may be employed for this purpose, and to make a nucleic acid molecule that has a sequence also found in a cell or organism. In lieu of such synthesis, the disclosed nucleic acid molecules may be used to define a pair of primers that can be used with the polymerase chain reaction to amplify and obtain any desired nucleic acid molecule or fragment.

“Identity,” as is well understood in the art, is a relationship between two or more polypeptide sequences or two or more nucleic acid molecule sequences, as determined by comparing the sequences. In the art, “identity” also means the degree of sequence relatedness between polypeptide or nucleic acid molecule sequences, as determined by the match between strings of such sequences. “Identity” can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York (1993); Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J. Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12:76-80 (1994); Birren el al., Genome Analysis, 1:543-559 (1997)). The BLASTX program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well-known Smith Waterman algorithm can also be used to determine identity.

Parameters for polypeptide sequence comparison typically include the following:

Algorithm: Needleman and Wunsch, J. Mol. Biol., 48:443-453 (1970)

Comparison matrix: BLOSSUM62 from Hentikoff and Hentikoff, Proc. Natl. Acad. Sci. USA, 89:10915-10919 (1992)

Gap Penalty: 12

Gap Length Penalty: 4

A program which can be used with these parameters is publicly available as the “gap” program from Genetics Computer Group, Madison, Wis. The above parameters along with no penalty for end gap are the default parameters for peptide comparisons.

Parameters for nucleic acid molecule sequence comparison include the following:

Algorithm: Needleman and Wunsch, J. Mol. Bio., 48:443-453 (1970)

Comparison matrix: matches—+10; mismatches=0

Gap Penalty: 50

Gap Length Penalty: 3

As used herein, “% identity” is determined using the above parameters as the default parameters for nucleic acid molecule sequence comparisons and the “gap” program from GCG, version 10.2.

The invention further relates to nucleic acid molecules that hybridize to nucleic acid molecules of the present invention. In particular, the invention relates to nucleic acid molecules that hybridize under stringent conditions to the above-described nucleic acid molecules. As used herein, the terms “stringent conditions” and “stringent hybridization conditions” mean that hybridization will generally occur if there is at least 95% and preferably at least 97% identity between the sequences. An example of stringent hybridization conditions is overnight incubation at 42° C. in a solution comprising 50% formamide, 5× SSC (150 mM NaCl, 15 mM trisodium citrate), 50 mM sodium phosphate (pH 7.6), 5× Denhardt's solution, 10% dextran sulfate, and 20 micrograms/milliliter denatured, sheared salmon sperm DNA, followed by washing the hybridization support in 0.1× SSC at approximately 65° C. Other hybridization and wash conditions are well known and are exemplified in Sambrook el al., Molecular Cloning: A Laboratory Manual, Second Edition, Cold Spring Harbor, N.Y. (1989), particularly Chapter 11.

In embodiments where nucleic acid sequences which when expressed are capable of selectively reducing the level of a FATB protein and/or FATB transcript, preferred nucleic acid sequences are selected from the groups consisting of (1) nucleic acid sequences with at least 50%, 60%, 70%, 80%, 90%, 95%, 97%, 98%, 99% or 100% sequence identity over the entire length of the nucleic acid molecule with a nucleotide sequence selected from the group consisting of SEQ ID NO: 2, SEQ ID NO: 3, SEQ ID NO: 4, SEQ [D NO: 5, SEQ ID NO: 6, SEQ ID NO: 7, SEQ ID NO: 8, complements thereof, and fragments of either; (2) nucleic acid molecules which contain sequences that are also found in a soybean FATB gene intron; and (3) nucleic acid molecules that exhibit sequences with at least 50%, 60%, 70%, 80%, 85%, 90%, 95%, 97%, 98%, 99% or 100% sequence identity over the entire length of the nucleic acid molecule with a nucleic acid molecule of (2).

One subset of the nucleic acid molecules of the invention includes fragment nucleic acid molecules. Fragment nucleic acid molecules may consist of significant portion(s) of, or indced most of, the nucleic acid molecules of the invention, such as those specifically disclosed. Alternatively, the fragments may comprise smaller oligonucleotides (having from about 15 to about 400 contiguous nucleotide residues and more preferably, about 15 to about 30 contiguous nucleotide residues, or about 50 to about 100 contiguous nucleotide residues, or about 100 to about 200 contiguous nucleotide residues, or about 200 to about 400 contiguous nucleotide residues, or about 275 to about 350 contiguous nucleotide residues). More preferably, the fragments may comprise small oligonucleotides having from about 15 to about 45 contiguous nucleotide residues, about 20 to about 45 contiguous nucleotide residues, about 15 to about 30 contiguous nucleotide residues, about 21 to about 30 contiguous nucleotide residues, about 21 to about 25 contiguous nucleotide residues, about 21 to about 25 contiguous nucleotide residues, about 19 to about 25 contiguous nucleotide residues, or about 21 contiguous nucleotides.

In another aspect, a fragment nucleic acid molecule has a nucleic acid sequence that is at least 15, 25, 50, or 100 contiguous nucleotides of a nucleic acid molecule of the present invention. In a preferred embodiment, the nucleic acid molecule has a nucleic acid sequence that is at least 15, 25, 50, or 100 contiguous nucleotides of a nucleic acid molecule having a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, and complements thereof.

A fragment of one or more of the nucleic acid molecules of the present invention may be a probe and specifically a PCR probe. A PCR probe is a nucleic acid molecule capable of initiating a polymerase activity while in a double-stranded structure with another nucleic acid molecule. Various methods for determining the structure of PCR probes and PCR techniques exist in the art. Computer generated searches using programs such as Primer3 (www-genome.wi.mit.edu/cgi-bin/primer/primer3.cgi), STSPipeline (www-genome.wi.mit.edu/cgi-bin/www-STS-Pipeline), or GeneUp (Pesole et al., BioTechniques 25:112-123 (1998)), for example, can be used to identify potential PCR primers.

Nucleic acid molecules or fragments thereof of the present invention are capable of specifically hybridizing to other nucleic acid molecules under certain circumstances. Nucleic acid molecules of the present invention include those that specifically hybridize to nucleic acid molecules having a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either. As used herein, two nucleic acid molecules are said to be capable of specifically hybridizing to one another if the two molecules are capable of forming an anti-parallel, double-stranded nucleic acid structure.

A nucleic acid molecule of the invention can also encode a homolog nucleic acid molecule. As used herein, a homolog nucleic acid molecule or fragment thereof is a counterpart nucleic acid molecule or fragment thereof in a second species (e.g., corn FATB intron I nucleic acid molecule is a homolog of Arabidopsis FATB intron I nucleic acid molecule). A homolog can also be generated by molecular evolution or DNA shuffling techniques, so that the molecule retains at least one functional or structure characteristic of the original polypeptide (see, for example, U.S. Pat. No. 5,811,238).

In another embodiment, the homolog is obtained from a plant selected from the group consisting of alfalfa, Arabidopsis, barley, Brassica campestris, oilseed rape, broccoli, cabbage, canola, citrus, cotton, garlic, oat, Allium, flax, an ornamental plant, jojoba, corn, peanut, pepper, potato, rapeseed, rice, rye, sorghum, strawberry, sugarcane, sugarbeet, tomato, wheat, poplar, pine, fir, eucalyptus, apple, lettuce, lentils, grape, banana, tea, turf grasses, sunflower, Phaseolus, crambe, mustard, castor bean, sesame, cottonseed, linseed, safflower, and oil palm. More particularly, a preferred homolog is obtained from a plant selected from the group consisting of canola, corn, Brassica campestris, oilseed rape, soybean, crambe, mustard, castor bean, peanut, sesame, cottonseed, linseed, rapeseed, safflower, oil palm, flax, and sunflower. In an even more preferred embodiment, the homolog is obtained from a plant selected from the group consisting of canola, rapeseed, corn, Brassica campestris, oilseed rape, soybean, sunflower, safflower, oil palm, and peanut.

In further embodiment, additional FATB introns may be obtained by any method by which additional introns may be identified. In a preferred embodiment, additional soybean FATB introns may be obtained by screening a soybean genomic library with a probe of either known exon or intron sequences of soybean FATB. The soybean FATB gene may then be cloned. In another preferred embodiment, additional soybean FATB introns may be obtained by a comparison between soybean genomic sequence and soybean cDNA sequence that allows identification of additional introns. In a more preferred embodiment, additional soybean FATB introns may be obtained by screening a soybean genomic library with a probe of either known exon or intron sequences of soybean FATB. The soybean FATB gene may then be cloned and confirmed and any additional introns may be identified by a comparison between soybean genomic sequence and soybean cDNA sequence. Additional introns may, for example without limitation, be amplified by PCR and used in an embodiment of the present invention.

In another preferred embodiment, an intron, such as for example, a soybean intron, may be cloned by alignment to an intron from another organism, such as, for example, Arabidopsis. In this embodiment, the location of an intron, for example, in an Arabidopsis amino acid sequence is identified. The Arabidopsis amino acid sequence, for example, may then be aligned, for example, with the soybean amino acid sequence, providing a prediction for the location, for example, of additional soybean FATB introns. Primers may be synthesized, for example, using the soybean FATB cDNA. The predicted introns may be synthesized, for example by PCR, using such primers. Such introns may be used in an embodiment of the present invention.

Plant Constructs and Plant Transformants

One or more of the nucleic acid molecules of the invention may be used in plant transformation or transfection. Exogenous genetic material may be transferred into a plant cell and the plant cell regenerated into a whole, fertile or sterile plant or plant part. Exogenous genetic material is any genetic material, whether naturally occurring or otherwise, from any source that is capable of being inserted into any organism.

In one embodiment of the invention, the expression level of a protein or transcript of one FATB gene family member is selectively reduced while leaving the level of a protein or transcript of a second FATB gene family member partially unaffected. In a preferred embodiment of the invention, the expression level of a protein or transcript in one FATB gene family member is selectively reduced while leaving the level of a protein or transcript of a second FATB gene family member substantially unaffected. In a highly preferred embodiment of the invention, the expression level of a protein or transcript of one FATB gene family member is selectively reduced while leaving the level of a protein or transcript of a second gene family member essentially unaffected.

As used herein, “partially unaffected” refers to a level of an agent such as a protein or mRNA transcript in which the level of the agent that is partially unaffected is within 80%, more preferably within 60%, and even more preferably within 50% of the level at which it is found in a cell or organism that lacks a nucleic acid molecule capable of selectively reducing another agent.

As used herein, “substantially unaffected” refers to a level of an agent such as a protein or mRNA transcript in which the level of the agent that is substantially unaffected is within 49%, more preferably within 35%, and even more preferably within 24% of the level at which it is found in a cell or organism that lacks a nucleic acid molecule capable of selectively reducing another agent.

As used herein, “essentially unaffected” refers to a level of an agent such as a protein or mRNA transcript that is either not altered by a particular event or altered only to an extent that does not affect the physiological function of that agent. In a preferred aspect, the level of an agent that is essentially unaffected is within 20%, more preferably within 10%, and even more preferably within 5% of the level at which it is found in a cell or organism that lacks a nucleic acid molecule capable of selectively reducing another agent.

In a more particularly preferred embodiment, a soybean plant of the present invention includes nucleic acid sequences which when expressed are capable of selectively reducing the expression level of a FATB protein and/or FATB transcript while overexpressing the level of a different protein and/or transcript. Preferably, the protein is selected from the group consisting of beta-ketoacyl-ACP synthase I, beta-ketoacyl-ACP synthase IV, and delta-9 desaturase, and the different transcript encodes an enzyme selected from the group consisting of beta-ketoacyl-ACP synthase I, beta-ketoacyl-ACP synthase IV, and delta-9 desaturase.

In embodiments where nucleic acid sequences which when expressed in a transformed plant are capable of selectively reducing the expression level of a FATB protein and/or FATB transcript, preferred nucleic acid sequences are selected from the groups consisting of (I) nucleic acid sequences with at least 50%, 60%, 70%, 80%, 85%, 90%, 95%, 97%, 98%, 99% or 100% sequence identity over the entire length of the nucleic acid molecule with a nucleotide sequence selected from the group consisting of SEQ ID NO: 2, SEQ ID NO:3, SEQ ID NO: 4, SEQ ID NO: 5, SEQ ID NO: 6, SEQ ID NO: 7, SEQ ID NO: 8, complements thereof, and fragments of either; (2) nucleic acid molecules which contain sequences that are also found in a soybean FATB gene intron; and (3) nucleic acid molecules that exhibit sequences with at least 50%, 60%, 70%, 80%, 85%, 90%, 95%, 97%, 98%, 99% or 100% sequence identity over the entire length of the nucleic acid molecule with a nucleic acid molecule of (2).

In a preferred embodiment, a soybean seed of the present invention has an oil composition that is 50% or greater oleic acid and 15% or less saturated fatty acids (including palmitic acid and stearic acid). In a more preferred embodiment, a soybean seed of the present invention has an oil composition that is 10% or less saturated fatty acids. As used herein, all % composition of oils within a plant or plant part such as a seed are determined by weight.

In a particularly preferred embodiment, a soybean seed of the present invention has an oil composition that is 9% or less, 8% or less, 7% or less, 6% or less, 5% or less, 4% or less, 3.6% or less, 3.5% or less, or 3.4% or less saturated fatty acids. In a more preferred embodiment, a soybean seed of the present invention has an oil composition that is a low saturate composition, and in another more preferred embodiment, a soybean seed of the present invention has an oil composition that is a zero saturate composition.

In another preferred embodiment a soybean seed of the present invention has an oil composition that is 50% or greater oleic acid, and between 10 and 15% saturated fatty acids. In a more preferred embodiment, a soybean seed of the present invention has an oil composition that is between 7 and 10% saturated fatty acids, between 5 and 8% saturated fatty acids, between 3.4 and 7% saturated fatty acids, between 3.5 and 7% saturated fatty acids, between 3.6 and 7% saturated fatty acids, between 2 and 4% saturated fatty acids, or less than 3.4% saturated fatty acids.

In another preferred embodiment of the present invention, a soybean seed of the present invention has an oil composition in which the level of palmitic acid is at least partially reduced, at least substantially reduced, or effectively eliminated. In another embodiment, a soybean seed of the present invention has an oil composition in which the level of stearic acid is at least partially reduced, at least substantially reduced, or effectively eliminated.

In embodiments where nucleic acid sequences which when expressed are capable of selectively reducing the expression level of a FATB protein and/or FATB transcript such that a soybean seed of the present invention has a low saturate or zero saturate oil composition that also contains 50% or greater oleic acid, the nucleic acid sequences are selected from the groups consisting of: (1) nucleic acid sequences with at least 50%, 60%, 70%, 80%, 85%, 90%, 95%, 97%, 98%, 99% or 100% sequence identity over the entire length of the nucleic acid molecule with a nuclcotide sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either; (2) nucleic acid molecules which contain sequences that are also found in a soybean FATB intron; and (3) nucleic acid molecules that exhibit sequences with at least 50%, 60%, 70%, 80%, 85%, 90%, 95%, 97%, 98%, 99% or 100% sequence identity over the entire length of the nucleic acid molecule with a nucleic acid molecule of (2).

Genetic material may also be obtained from other species, for example monocotyledons or dicotyledons, including, but not limited to canola, corn, soybean, Arabidopsis, Phaseolus, peanut, alfalfa, wheat, rice, oat, sorghum, rapeseed, rye, barley, millet, fescue, perennial ryegrass, sugarcane, cranberry, papaya, banana, safflower, oil palm, flax, muskmelon, apple, cucumber, dendrobium, gladiolus, chrysanthemum, liliacea, cotton, eucalyptus, sunflower, Brassica campestris, oilseed rape, turfgrass, sugarbeet, coffee and dioscorea (Christou, INO: Particle Bombardment for Genetic Engineering of Plants, Biotechnology Intelligence Unit. Academic Press, San Diego, Calif. (1996)), with canola, corn, Brassica campestris, oilseed rape, rapeseed, soybean, crambe, mustard, castor bean, peanut, sesame, cottonseed, linseed, safflower, oil palm, flax, and sunflower preferred, and canola, rapeseed, corn, Brassica campestris, oilseed rape, soybean, sunflower, safflower, oil palms, and peanut more preferred. In a more preferred embodiment, canola genetic material is transferred into canola. In another more preferred embodiment, oilseed rape genetic material is transferred into oilseed rape. In another particularly preferred embodiment, soybean genetic material is transferred into soybean.

The levels of products such as transcripts or proteins may be increased or decreased throughout an organism such as a plant or localized in one or more specific organs or tissues of the organism. For example the levels of products may be increased or decreased in one or more of the tissues and organs of a plant including without limitation: roots, tubers, stems, leaves, stalks, fruit, berries, nuts, bark, pods, seeds and flowers. A preferred organ is a seed.

Exogenous genetic material may be transferred into a host cell by the use of a DNA vector or construct designed for such a purpose. Design of such a vector is generally within the skill of the art (See, e.g., Plant Molecular Biology: A Laboratory Manual, Clark (ed.), Springer, New York (1997)).

A construct or vector may include a plant promoter to express a nucleic acid molecule of choice. In a preferred embodiment, any nucleic acid molecules described herein can be operably linked to a promoter region which functions in a plant cell to cause the production of an mRNA molecule. For example, any promoter that functions in a plant cell to cause the production of an mRNA molecule, such as those promoters described herein, without limitation, can be used. In a preferred embodiment, the promoter is a plant promoter.

A number of promoters that are active in plant cells have been described in the literature. These include, but are not limited to, the nopaline synthase (NOS) promoter (Ebert et al., Proc. Natl Acad Sci. (U.S.A.) 84:5745-5749 (1987)), the octopine synthase (OCS) promoter (which is carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987)) and the CaMV 35S promoter (Odell et al., Nature 313:810-812 (1985)), the figwort mosaic virus 35S-promoter (U.S. Pat. No. 5,378,619), the light-inducible promoter from the small subunit of ribulose-1,5-bis-phosphate carboxylase (ssRUBISCO), the Adh promoter (Walker et al., Proc. Natl. Acad Sci. (U.S.A.) 84:6624-6628 (1987)), the sucrose synthase promoter (Yang et al., Proc. Natl. Acad Sci. (US.A.) 87:4144-4148 (1990)), the R gene complex promoter (Chandler et al., The Plant Cell 1:1175-1183(1989)) and the chlorophyll a/b binding protein gene promoter. These promoters have been used to create DNA constructs that have been expressed in plants; see, e.g., PCT publication WO 84/02913. The CaMV 35S promoters are preferred for use in plants. Promoters known or found to cause transcription of DNA in plant cells can be used in the invention.

Particularly preferred promoters can also be used to express a nucleic acid molecule of the present invention in seeds or fruits. Indeed, in a preferred embodiment, the promoter used is a seed specific promoter. Examples of such promoters include the 5′ regulatory regions from such genes as napin (Kridl et al., Seed Sci. Res. 1:209-219 (1991)), phaseolin (Bustos et al., Plant Cell, 1(9):839-853 (1989)), soybean trypsin inhibitor (Riggs et al., Plant Cell 1(6):609-621 (1989)), ACP (Baerson et al., Plant Mol. Biol., 22(2):255-267 (1993)), stearoyl-ACP desaturase (Slocombe et al., Plant Physiol. 104(4):167-176 (1994)), soybean a subunit of b-conglycinin (soy 7s, (Chen et al., Proc. Natl. Acad. Sci., 83:8560-8564 (1986))), and oleosin (see, for example, Hong et al., Plant Mol. Biol., 34(3):549-555 (1997)). Further examples include the promoter for β-conglycinin (Chen et al., Dev. Genet. 10: 112-122 (1989)) and the promoter for FAE (PCT Publication WO 01/11061). Preferred promoters for expression in the seed are 7S and napin promoters.

Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435; and 4,633,436. In addition, a tissue specific enhancer may be used (Fromm et al., The Plant Cell 1:977-984 (1989)).

Constructs or vectors may also include, with the region of interest, a nucleic acid sequence that acts, in whole or in part, to terminate transcription of that region. A number of such sequences have been isolated, including the Tr7 3′ sequence and the NOS 3′ sequence (Ingelbrecht et al., The Plant Cell 1:671-680 (1989); Bevan el al., Nucleic Acids Res. 11:369-385 (1983)). Regulatory transcript termination regions can be provided in plant expression constructs of this invention as well. Transcript termination regions can be provided by the DNA sequence encoding the gene of interest or a convenient transcription termination region derived from a different gene source, for example, the transcript termination region that is naturally associated with the transcript initiation region. The skilled artisan will recognize that any convenient transcript termination region that is capable of terminating transcription in a plant cell can be employed in the constructs of the present invention.

A vector or construct may also include regulatory elements. Examples of such include the Adh intron 1 (Callis et al., Genes and Develop. 1:1183-1200 (1987)), the sucrose synthase intron (Vasil et al., Plant Physiol. 91:1575-1579 (1989)) and the TMV omega element (Gallie et al., The Plant Cell 1:301-311 (1989)). These and other regulatory elements may be included when appropriate.

A vector or construct may also include a selectable marker. Selectable markers may also be used to select for plants or plant cells that contain the exogenous genetic material. Examples of such include, but are not limited to: a neo gene (Potrykus et al., Mol. Gen. Genet. 199:183-188 (1985)), which codes for kanamycin resistance and can be selected for using kanamycin, RptII, G418, hpt; a bar gene which codes for bialaphos resistance; a mutant EPSP synthase gene (Hinchee et al., Bio/Technology 6:915-922 (1988); Reynaerts et al., Selectable and Screenable Markers. In Gelvin and Schilperoort. Plant Molecular Biology Manual, Kluwer, Dordrecht (1988); Reynaerts et al., Selectable and screenable markers. In Gelvin and Schilperoort. Plant Molecular Biology Manual, Kluwer, Dordrecht (1988)), aadA (Jones et al, Mol. Gen. Genet. (1987)), which encodes glyphosate resistance; a nitrilase gene which confers resistance to bromoxynil (Stalker et al., J. Biol. Chem. 263:6310-6314 (1988)); a mutant acetolactate synthase gene (ALS) which confers imidazolinone or sulphonylurea resistance (European Patent Application 154,204 (Sep. 11, 1985)), ALS (D'Halluin et al., Bio/Technology 10: 309-314 (1992)), and a methotrexate resistant DHFR gene (Thillet et al., J. Biol. Chem. 263:12500-12508 (1988)).

A vector or construct may also include a screenable marker. Screenable markers may be used to monitor expression. Exemplary screenable markers include: a β-glucuronidase or uidA gene (GUS) which encodes an enzyme for which various chromogenic substrates are known (Jefferson, Plant Mol Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 25 11:263-282 (1988)); a β-lactamase gene (Sutcliffe etal., Proc. Natl. Acad Sci. (U.S.A.) 75:3737-3741 (1978)), a gene which encodes an enzyme for which various chrornogenic substrates are known (e.g., PADAC, a chromogenic cephalosporin); a luciferase gene (Ow et al., Science 234:856-859 (1986)); a xylE gene (Zukowsky et al., Proc. Natl. Acad. Sci. (U.S.A.) 80:1101-1105 (1983)) which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to melanin; an α-galactosidase, which will turn a chromogenic α-galactose substrate.

Included within the terms “selectable or screenable marker genes” are also genes that encode a secretable marker whose secretion can be detected as a means of identifying or selecting for transformed cells. Examples include markers that encode a secretable antigen that can be identified by antibody interaction, or even secretable enzymes that can be detected catalytically. Secretable proteins fall into a number of classes, including small, diffusible proteins that are detectable, (e.g., by ELISA), small active enzymes that are detectable in extracellular solution (e.g., α-amylase, β-lactamase, phosphinothricin transferase), or proteins that are inserted or trapped in the cell wall (such as proteins that include a leader sequence such as that found in the expression unit of extension or tobacco PR-S). Other possible selectable and/or screenable marker genes will be apparent to those of skill in the art.

It is understood that two or more nucleic molecules of the present invention may be introduced into a plant using a single construct and that construct can contain more than one promoter. In embodiments where the construct is designed to express two nucleic acid molecules, it is preferred that the two promoters are (i) two constitutive promoters, (ii) two seed-specific promoters, or (iii) one constitutive promoter and one seed-specific promoter. Preferred seed-specific and constitutive promoters are a napin and a 7S promoter, respectively. Illustrative combinations are set forth in Example 5. It is understood that two or more of the nucleic molecules may be physically linked and expressed utilizing a single promoter, preferably a seed-specific or constitutive promoter.

It is further understood that two or more nucleic acids of the present invention may be introduced into a plant using two or more different constructs. Alternatively, two or more nucleic acids of the present invention may be introduced into two different plants and the plants may be crossed to generate a single plant expressing two or more nucleic acids. In an RNAi embodiment, it is understood that the sense and antisense strands may be introduced into the same plant on one construct or two constructs. Alternatively, the sense and antisense strands may be introduced into two different plants and the plants may be crossed to generate a single plant expressing both sense and antisense strands.

Any of the nucleic acid molecules and constructs of the invention may be introduced into a plant or plant cell in a permanent or transient manner. Preferred nucleic acid molecules and constructs of the present invention are described above in the Detailed Description, and in the Examples. Another embodiment of the invention is directed to a method of producing transgenic plants which generally comprises the steps of selecting a suitable plant or plant cell, transforming the plant or plant cell with a recombinant vector, and obtaining a transformed host cell.

In a preferred embodiment the plant or cell is, or is derived from, a plant involved in the production of vegetable oils for edible and industrial uses. Especially preferred are temperate oilseed crops. Plants of interest include, but are not limited to, rapeseed (canola and High Erucic Acid varieties), maize, soybean, crambe, mustard, castor bean, peanut, sesame, cotton, linseed, safflower, oil palm, flax, sunflower, and coconut. The invention is applicable to monocotyledonous or dicotyledonous species alike, and will be readily applicable to new and/or improved transformation and regulatory techniques.

Methods and technology for introduction of DNA into plant cells are well known to those of skill in the art, and virtually any method by which nucleic acid molecules may be introduced into a cell is suitable for use in the present invention. Non-limiting examples of suitable methods include: chemical methods; physical methods such as microinjection, electroporation, the gene gun, microprojectile bombardment, and vacuum infiltration; viral vectors; and receptor-mediated mechanisms. Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

Agrobacteriuni-mediated transfer is a widely applicable system for introducing genes into plant cells. See, e.g., Fraley et al., Bio/Technology 3:629-635 (1985); Rogers et al., Methods Enzymol. 153:253-277 (1987). The region of DNA to be transferred is defined by the border sequences and intervening DNA is usually inserted into the plant genome. Spielmann et al., Mol. Gen. Genet. 205:34 (1986). Modern Agrobacterium transformation vectors are capable of replication in E. coli as well as Agrobacterium, allowing for convenient manipulations. Klee et al., In: Plant DNA Infectious Agents, Hohn and Schell (eds.), Springer-Verlag, New York, pp. 179-203 (1985).

The regeneration, development and cultivation of plants from single plant protoplast transformants or from various transformed explants is well known in the art. See generally, Maliga et al., Methods in Plant Molecular Biology, Cold Spring Harbor Press (1995); Weissbach and Weissbach, In: Methods for Plant Molecular Biology, Academic Press, San Diego, Calif. (1988). Plants of the present invention can be part of or generated from a breeding program, and may also be reproduced using apomixis. Methods for the production of apomictic plants are known in the art. See, e.g., U.S. Pat. No. 5,811,636.

Cosuppression is the reduction in expression levels, usually at the level of RNA, of a particular endogenous gene or gene family by the expression of a homologous sense construct that is capable of transcribing MRNA of the same strandedness as the transcript of the endogenous gene (Napoli et al., Plant Cell 2:279-289 (1990); van der Krol et al., Plant Cell 2:291-299 (1990)). Cosuppression may result from stable transformation with a single copy nucleic acid molecule that is homologous to a nucleic acid sequence found with the cell (Prolls and Meyer, Plant J. 2:465-475 (1992)) or with multiple copies of a nucleic acid molecule that is homologous to a nucleic acid sequence found with the cell (Mittlesten et al., Mol. Gen. Genet. 244.325-330 (1994)). Genes, even though different, linked to homologous promoters may result in the cosuppression of the linked genes (Vaucheret, CR. Acad Sci. III 316:1471-1483 (1993); Flavell, Proc. Natl. Acad. Sci. (U.S.A.) 91:3490-3496 (1994)); van Blokland et al., Plant J. 6:861-877 (1994); Jorgensen, Trends Biotechnol. 8:340-344 (1990); Meins and Kunz, In: Gene Inactivation and Homologous Recombination in Plants, Paszkowski (ed.), pp. 335-348, Kluwer Academic, Netherlands (1994)) (Kinney, Induced Mutations and Molecular Techniques for Crop Improvement, Proceedings of a Symposium 19-23 Jun. 1995 jointly organized by IAEA and FA)), pages 101-113 (IAEA-SM 340-49).

It is understood that one or more of the nucleic acids of the invention may be introduced into a plant cell and transcribed using an appropriate promoter with such transcription resulting in the cosuppression of an endogenous protein.

Antisense approaches are a way of preventing or reducing gene function by targeting the genetic material (Mol et al., FEBS Lett. 268:427-430 (1990)). The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes (Hiatt et al., In: Genetic Engineering, Setlow (ed.), Vol. 11, New York: Plenum 49-63 (1989)).

Antisense RNA techniques involve introduction of RNA that is complementary to the target mRNA into cells, which results in specific RNA:RNA duplexes being formed by base pairing between the antisense substrate and the target mRNA (Green et al., Annu. Rev. Biochem. 55:569-597 (1986)). Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression (Takayama and Inouye, Crit. Rev. Biochem. Mol. Biol. 25:155-184 (1990)). An antisense vector is constructed by standard procedures and introduced into cells by methods including but not limited to transformation, transfection, electroporation, microinjection, and infection. The type of transformation and choice of vector will determine whether expression is transient or stable. The promoter used for the antisense gene may influence the level, timing, tissue, specificity, or inducibility of the antisense inhibition.

It has been reported that the introduction of double-stranded RNA into cells may also be used to disrupt the function of an endogenous gene. (Fire et al., Nature 391: 806-811 (1998)). Such disruption has been demonstrated, for example, in Caenorhabditis elegans and is often referred to as RNA interference, or RNAi. (Fire et al., Nature 391: 806-811 (1998)). The disruption of gene expression in C. elegans by double-stranded RNA has been reported to induce suppression by a post-transcriptional mechanism. (Montgomery et al., Proc. Natl. Acad. Sci. 95:15502-15507 (1998)). Evidence of gene silencing by double-stranded RNA has also been reported for plants. (Waterhouse et al., Proc. Natl. Acad. Sci. 95: 13959-13964 (1998)). See also Plasterk, Science 296: 1263-1265 (2002); Zamore, Science 296: 1265-1269 (2002).

An intron-spliced hairpin structure reportedly may also be used to effect post-transcriptional gene suppression. (Smith et al., Nature 407: 319-320 (2000)). Reports indicate that post-transcriptional gene silencing can be induced with almost 100% efficiency by the use of intron-spliced RNA with a hairpin structure. (Smith et al., Nature 407: 319-320 (2000)). Representative methods for effecting RNA silencing are set forth in U.S. Application, Attorney Docket No. 16518.069, entitled “Intron Double Stranded RNA Constructs And Uses Thereof,” naming JoAnne Fillatti as inventor, filed concurrently herewith.

It is understood that one or more of the nucleic acids of the invention may be modified in order to effect RNAi or another mode of post-transcriptional gene suppression.

The present invention also provides for parts of the plants, particularly reproductive or storage parts. Plant parts, without limitation, include seed, endospermn, ovule, pollen, roots, tubers, stems, leaves, stalks, fruit, berries, nuts, bark, pods, seeds and flowers. In a particularly preferred embodiment of the present invention, the plant part is a seed.

The present invention also provides a container of over 10,000, more preferably 20,000, and even more preferably 40,000 seeds where over 10%, more preferably 25%, more preferably 50% and even more preferably 75% or 90% of the seeds are seeds derived from a plant of the present invention.

The present invention also provides a container of over 10 kg, more preferably 25 kg, and even more preferably 50 kg seeds where over 10%, more preferably 25%, more preferably 50% and even more preferably 75% or 90% of the seeds are seeds derived from a plant of the present invention.

Any of the plants or parts thereof of the present invention may be processed to produce a feed, meal, protein, or oil preparation. A particularly preferred plant part for this purpose is a seed. In a preferred embodiment the feed, meal, protein or oil preparation is designed for livestock animals or humans, or both. Methods to produce feed, meal, protein and oil preparations are known in the art. See, for example, U.S. Pat. Nos. 4,957,748, 5,100,679, 5,219,596, 5,936,069, 6,005,076, 6,146,669, and 6,156,227. In a preferred embodiment, the protein preparation is a high protein preparation. Such a high protein preparation preferably has a protein content of greater than 5% w/v, more preferably 10% w/v, and even more preferably 15% w/v. In a preferred oil preparation, the oil preparation is a high oil preparation with an oil content derived from a plant or part thereof of the present invention of greater than 5% w/v, more preferably 10% w/v, and even more preferably 15% w/v. In a preferred embodiment the oil preparation is a liquid and of a volume greater than 1, 5, 10 or 50 liters. The present invention provides for oil produced from plants of the present invention or generated by a method of the present invention. Such an oil may exhibit enhanced oxidative stability. Also, such oil may be a minor or major component of any resultant product. Moreover, such oil may be blended with other oils. In a preferred embodiment, the oil produced from plants of the present invention or generated by a method of the present invention. constitutes greater than 0.5%, 1%, 5%, 10%, 25%, 50%, 75% or 90% by volume or weight of the oil component of any product. In another embodiment, the oil preparation may be blended and can constitute greater than 10%, 25%, 35%, 50% or 75% of the blend by volume. Oil produced from a plant of the present invention can be admixed with one or more organic solvents or petroleum distillates.

In one embodiment, an oil of the present invention has an oil composition that is 50% or greater oleic acid and 15% or less saturated fatty acids. In another embodiment, an oil of the present invention has an oil composition that is 10% or less saturated fatty acids. In another embodiment, an oil of the present invention has an oil composition that is 9% or less saturated fatty acids, 8% or less saturated fatty acids, 7% or less saturated fatty acids, 6% or less saturated fatty acids, 5% or less saturated fatty acids, 4% or less saturated fatty acids, 3.6% or less saturated fatty acids, 3.5% or less saturated fatty acids, or 3.4% or less saturated fatty acids. In a more preferred embodiment, an oil of the present invention has a low saturate oil composition, and in another preferred embodiment, an oil of the present invention has a zero saturate oil composition.

In another preferred embodiment, an oil of the present invention has an oil composition that is 50% or greater oleic acid, and between 10 and 15% saturated fatty acids. In a more preferred embodiment, an oil of the present invention has an oil composition that is between 7 and 10% saturated fatty acids, between 5 and 8% saturated fatty acids, between 3.4 and 7% saturated fatty acids, between 3.5 and 7% saturated fatty acids, between 3.6 and 7% saturated fatty acids, between 2 and 4% saturated fatty acids, or less than 3.4% saturated fatty acids.

In another preferred embodiment, an oil of the present invention has an oil composition in which the level of palmitic acid is at least partially reduced, at least substantially reduced, or effectively eliminated. In another embodiment, an oil of the present invention has an oil composition in which the level of stearic acid is at least partially reduced, at least substantially reduced, or effectively eliminated.

In embodiments where nucleic acid sequences which when expressed are capable of selectively reducing the expression level of a protein and/or transcript encoded by a FATB gene such that an oil of the present invention has an oil composition that is 50% or greater oleic acid, and 10% or less saturated fatty acids, preferably 5% or less saturated fatty acids, preferably 3.6% or less saturated fatty acids, preferably 3.5% or less saturated fatty acids, and more preferably 3.4% or less saturated fatty acids, the nucleic acid sequences are selected from the groups consisting of: (I) nucleic acid sequences with at least 50%, 60%, 70%, 80%, 85%, 90%, 95%, 97%, 98%, 99% or 100% sequence identity over the entire length of the nucleic acid molecule with a nucleotide sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either; (2) nucleic acid molecules which contain sequences that are also found in a soybean FATB gene intron; and (3) nucleic acid molecules that exhibit sequences with at least 50%, 60%, 70%, 80%, 85%, 90%, 95%, 97%, 98%, 99% or 100% sequence identity over the entire length of the nucleic acid molecule with a nucleic acid molecule of (2).

Computer Readable Medium

The nucleotide sequence provided in SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, or a nucleotide sequence at least 50%, 60%, or 70% identical, preferably 80%, 85% identical, or especially preferably 90%, or 95% identical, or particularly highly preferably 97%, 98%, or 99% identical to the sequence provided in SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of either, can be “provided” in a variety of media to facilitate use. Such a medium can also provide a subset thereof in a form that allows a skilled artisan to examine the sequences.

In one application of this embodiment, a nucleotide sequence of the present invention can be recorded on computer readable media. As used herein, “computer readable media” refers to any medium that can be read and accessed directly by a computer. Such media include, but are not limited to: magnetic storage media, such as floppy disk, hard disk, storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising a computer readable medium having recorded thereon a nucleotide sequence of the present invention.

As used herein, “recorded” refers to a process for storing information on computer readable medium. A skilled artisan can readily adopt any of the presently known methods for recording information on computer readable media to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable media. The sequence information can be represented in a word processing text file, formatted in commercially-available software such as Word Perfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain computer readable media having recorded thereon the nucleotide sequence information of the present invention.

By providing one or more nucleotide sequences of the present invention, a skilled artisan can routinely access the sequence information for a variety of purposes. Computer software is publicly available which allows a skilled artisan to access sequence information provided in a computer readable medium. Software which implements the BLAST (Altschul et al., J. Mol. Biol. 215: 403-410 (1990)) and BLAZE (Brutlag et al., Comp. Chem. 17:203-207 (1993)) search algorithms on a Sybase system can be used to identify non-coding regions and other nucleic acid molecules of the present invention within the genome that contain homology to non-coding regions from other organisms. Such non-coding regions may be utilized to affect the expression of commercially important proteins such as enzymes used in amino acid biosynthesis, metabolism, transcription, translation, RNA processing, nucleic acid and protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecules of the present invention. As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems is suitable for use in the present invention.

As indicated above, the computer-based systems of the present invention comprise a data storage means having stored therein a nucleotide sequence of the present invention and the necessary hardware means and software means for supporting and implementing a search means. As used herein, “data storage means” refers to memory that can store nucleotide sequence information of the present invention, or a memory access means which can access manufactures having recorded thereon the nucleotide sequence information of the present invention. As used herein, “search means” refers to one or more programs which are implemented on the computer-based system to compare a target sequence or target structural motif with the sequence information stored within the data storage means. Search means are used to identify fragments or regions of the sequence of the present invention that match a particular target sequence or target motif A variety of known algorithms are disclosed publicly and a variety of commercially available software for conducting search means are available and can be used in the computer-based systems of the present invention. Examples of such software include, but are not limited to, MacPattem (EMBL), BLASTIN, and BLASTIX (NCBIA). One of the available algorithms or implementing software packages for conducting homology searches can be adapted for use in the present computer-based systems.

The most preferred sequence length of a target sequence is from about 10 to 100 amino acids or from about 30 to 300 nucleotide residues. However, it is well recognized that during searches for commercially important fragments of the nucleic acid molecules of the present invention, such as sequence fragments involved in gene expression and protein processing, the target sequence may be of shorter length.

As used herein, “a target structural motif,” or “target motif” refers to any rationally selected sequence or combination of sequences in which the sequences are chosen based on a three-dimensional configuration which is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures, and inducible expression elements (protein binding sequences).

Thus, the present invention further provides an input means for receiving a target sequence, a data storage means for storing the target sequences of the present invention sequence identified using a search means as described above, and an output means for outputting the identified homologous sequences. A variety of structural formats for the input and output means can be used to input and output information in the computer-based systems of the present invention. A preferred format for an output means ranks fragments of the sequence of the present invention by varying degrees of homology to the target sequence or target motif. Such presentation provides a skilled artisan with a ranking of sequences which contain various amounts of the target sequence or target motif and identifies the degree of homology contained in the identified fragment.

A variety of comparing means can be used to compare a target sequence or target motif with the data storage means to identify sequence fragments sequence of the present invention. For example, implementing software that implement the BLAST and BLAZE algorithms (Altschul et al., J. Mol. Biol. 215:403-410 (1990)) can be used to identify non-coding regions within the nucleic acid molecules of the present invention. A skilled artisan can readily recognize that any one of the publicly available homology search programs can be used as the search means for the computer-based systems of the present invention.

The following examples are illustrative and not intended to be limiting in any way.

### EXAMPLES

### Example 1

### Cloning of FATB Thioesterasc Genomic Sequences

Leaf tissue is obtained from Asgrow soy variety A3244, ground up in liquid nitrogen and stored at −80° C. until use. Six ml of SDS Extraction buffer (650 ml sterile ddH₂0, 100 ml 1M Tris-Cl pH 8, 100 ml 0.25M EDTA, 50 ml 20% SDS, 100 ml 5M NaCl, 4 μl beta-mercaptoethanol) is added to 2 ml of frozen/ground leaf tissue, and the mixture is incubated at 65° C. for 45 minutes. The sample is shaken every 15 minutes. 2 ml of ice-cold 5M potassium acetate is added to the sample, the sample is shaken, and then is incubated on ice for 20 minutes. 3 ml of CHCl₃ is added to the sample and the sample is shaken for 10 minutes.

The sample is centrifuged at 10,000 rpm for 20 minutes and the supernatant is collected. 2 ml of isopropanol is added to the supernatant and mixed. The sample is then centrifuged at 10,000 rpm for 20 minutes and the supernatant is drained. The pellet is resuspended in 200 μl RNase and incubated at 65° C. for 20 minutes. 300 μl ammonium acetate/isopropanol (1:7) is added and mixed. The sample is then centrifuged at 10,000 rpm for 15 minutes and the supernatant was discarded. The pellet is rinsed with 500 μl 80% ethanol and allowed to air dry. The pellet of genomic DNA is then resuspended in 200 μl T10E1 (10 mM Tris: 1 mM EDTA).

In a first method, a soy FATB cDNA sequence is used to design six oligonucleotides that span the gene: F1 (SEQ ID NO: 11), F2 (SEQ ID NO: 12), F3 (SEQ ID NO: 13), R1 (SEQ ID NO: 14), R2 (SEQ ID NO: 15), and R3 (SEQ ID NO: 16). The oligonucleotides are used in pairs for PCR amplification from the isolated soy genomic DNA: pair 1 (F1+R1), pair 2 (F1+R2), pair 3 (F1+R3), pair 4 (F2+R1), pair 5 (F2 +R2), pair 6 (F2+R3), pair 7 (F3+R1), and pair 8 (F3+R2). The PCR amplifications is carried out as follows: 1 cycle, 95° C. for 10 minutes; 40 cycles, 95° C. for 1 minutes, 58° C. for 30 sec, 72° C. for 55 sec; 1 cycle, 72° C. for 7 minutes. Three positive fragments are obtained, specifically from primer pairs 3, 6, and 7. Each fragment is cloned into vector pCR2.1 (Invitrogen). Cloning is successful only for genomic fragment #3, which is confirmed and sequenced (SEQ ID NO: 10).

Three introns are identified in the soybean FATB gene by comparison of the genomic sequence to the cDNA sequence: intron I (SEQ ID NO: 2) spans base 106 to base 214 of the genomic sequence (SEQ ID NO: 10) and is 109 bp in length; intron II (SEQ ID NO: 3) spans base 289 to base 1125 of the genomic sequence (SEQ ID NO: 10) and is 837 bp in length; and intron III (SEQ ID NO: 4) spans base 1635 to base 1803 of the genomic sequence (SEQ ID NO: 10) and is 169 bp in length.

In a second method, the Arabidopsis thaliana FATB cDNA and A. thaliana FATB genomic sequence are aligned with the soy FATB cDNA and the potential locations of soy FATB introns are determined. Oligonucleotides are synthesized for sequences flanking the putative soy introns, and genomic DNA is amplified using appropriate primer pairs. Four additional introns are identified in the soybean FATB gene by comparison of the amplified genomic sequences to the cDNA sequence. These four soy intron sequences are combined with the soy cDNA sequence and the three previously isolated soy intron sequences to generate a genomic sequence of the FATB gene (SEQ ID NO: 1). The four new introns isolated are as follows: primers F1 and R1 yield intron IV (SEQ ID NO: 5 ), which spans base 1939 to base 2463 of the genomic sequence (SEQ ID NO: 1) and is 525 bp in length; primers F2 and R2 yield intron V (SEQ ID NO: 6), which spans base 2578 to base 2966 of the genomic sequence (SEQ ID NO: 1) and is 389 bp in length; primers F3 and R3 yield intron VI (SEQ ID NO: 7 ) spans base 3140 to base 3245 of the genomic sequence (SEQ ID NO: 1) and is 106 bp in length and intron VII (SEQ ID NO: 8) which spans base 3314 to base 3395 of the genomic sequence (SEQ ID NO: 1) and is 82 bp in length.

### Example 2

### Plant Expression Constructs

A soybean FATB intron II sequence (SEQ ID NO: 3) is amplified via PCR using the partial FATB cloned genomic DNA sequence (SEQ ID NO: 10) as a template and primers 18133 (SEQ ID NO: 17) and 18134 (SEQ ID NO: 18). PCR amplification is carried out as follows: 1 cycle, 95° C. for 10 minutes; 25 cycles, 95° C. for 30 sec, 62° C. for 30 sec, 72° C. for 30 sec; 1 cycle, 72° C. for 7 minutes.

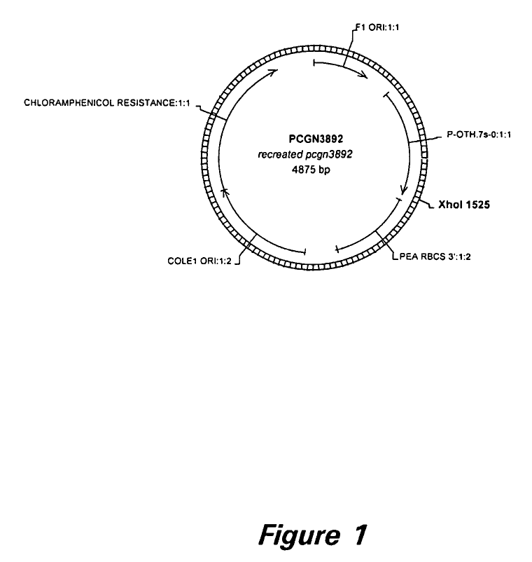

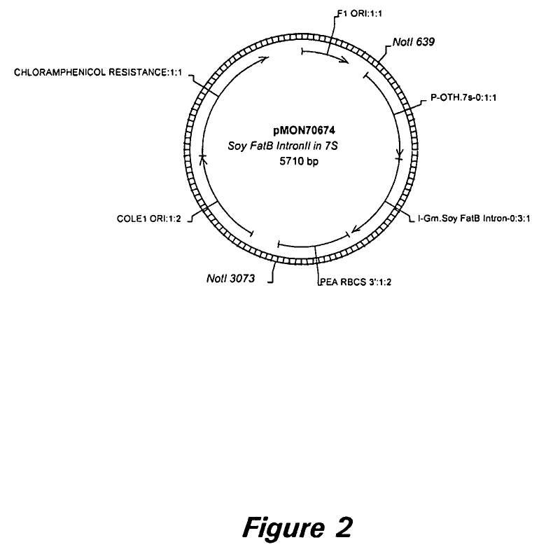

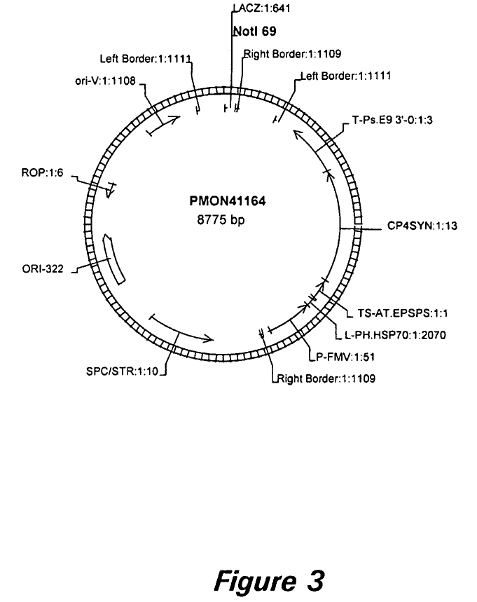

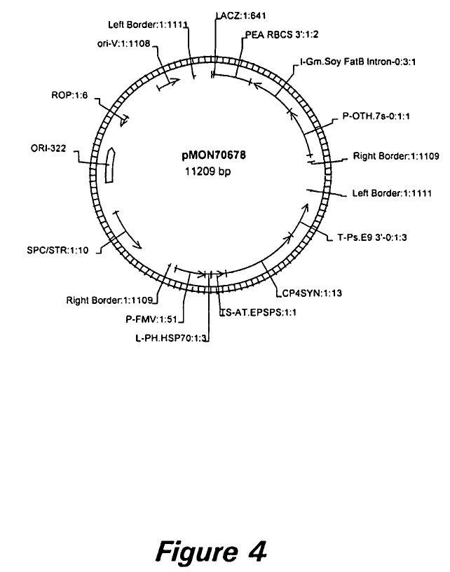

PCR amplification resulted in a product (SEQ ID NO: 19) that is 854 bp long. The PCR product is cloned directly into the expression cassette pCGN3892 (FIG. 1) in sense orientation, by way of XhoI sites engineered onto the 5′ ends of the PCR primers, to form pMON70674 (FIG. 2). Vector pCGN3892 contains the soybean 7S promoter and a pea RBCS 3′. pMON70674 is then cut with NotI and ligated into pMON41164, a vector that contains the CP4 gene regulated by the FMV promoter (FIG. 3). The resulting gene expression construct, pMON70678 (FIG. 4), is used for transformation of soybean using Agrobacterium methods as described herein.

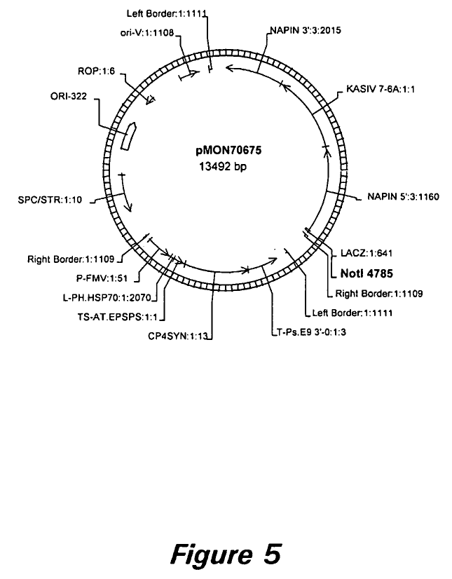

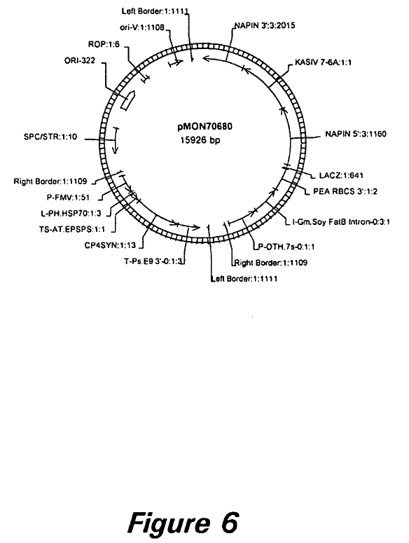

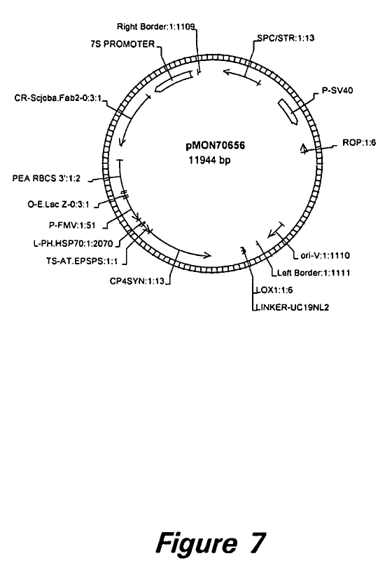

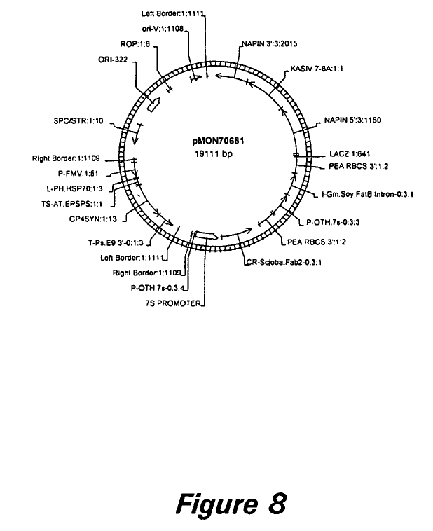

Two other expression constructs containing the soybean FATB intron II sequence (SEQ ID NO: 3) are created. pMON70674 is cut with NotI and ligated into pMON70675 (FIG. 5) which contains the CP4 gene regulated by the FMV promoter and the KAS IV gene regulated by the napin promoter. The resulting expression construct, pMON70680 (FIG. 6), is used for transformation of soybean using Agrobacterium methods as described herein. The expression vector pMON70680 is then cut with SnaBI and ligated with a gene fusion of the jojoba delta-9 desaturase gene in the sense orientation regulated by the 7S promoter (pMON70656; FIG. 7). The resulting expression construct, pMON70681 (FIG. 8), is used for transformation of soybean using Agrobacterium methods as described herein.

Other soybean FATB intron sequences, such as SEQ ID NO: 3, SEQ ID NO: 4, SEQ ID NO: 5, SEQ ID NO: 6, SEQ ID NO: 7, or SEQ ID NO: 8, are cloned in a similar manner.

Appropriate primers are designed based on the intron sequence desired. These primer pairs are used to amplify an intron from the FATB genomic sequence. The amplified intron is ligated into the desired expression vector and the construct is transformed into soybean.

### Example 3

### Plant Transformation And Analysis

Linear DNA fragments containing the expression constructs of the soybean FATB introns are stably introduced into soybean (Asgrow variety A3244) by the method of Martinell et al., U. S. Pat. No. 6,384,301. Transformed soybean plants are identified by selection on media containing glyphosate.

Fatty acid compositions are analyzed from seed of soybean lines transformed with the intron expression constructs using gas chromatography. R₁ single seed oil compositions of plants harboring pMON70678 demonstrate that the saturated and unsaturated fatty acid compositions are altered in the oil of seeds from transgenic soybean lines as compared to those of the seed from non-transformed soybean (Table 1). In particular, 16:0 is reduced in transgenic seeds. Selections can be made from such lines depending on the desired relative fatty acid composition. In addition, since each of the introns is able to modify the levels of each fatty acid to varying extents, it is contemplated that combinations of introns can be used depending on the desired compositions.

TABLE 1
R1 single seed

| data | | Fatty Acids | | | | |
|---|---|---|---|---|---|---|
| Construct | Event | 16:0 | 18:0 | 18:1 | 18:2 | 18:3 |
| PMON70678 | GM_A31349 | 7.7 | 5.0 | 17.4 | 62.2 | 7.7 |
| PMON70678 | GM_A31349 | 7.8 | 4.6 | 18.2 | 61.9 | 7.3 |
| PMON70678 | GM_A31349 | 7.9 | 5.5 | 17.3 | 60.4 | 8.3 |
| PMON70678 | GM_A31349 | 7.9 | 5.0 | 17.3 | 60.6 | 8.6 |
| PMON70678 | GM_A31349 | 8.2 | 5.5 | 15.6 | 61.8 | 8.3 |
| PMON70678 | GM_A31342 | 8.4 | 6.8 | 12.4 | 63.1 | 9.0 |
| PMON70678 | GM_A31342 | 8.7 | 5.3 | 15.9 | 62.7 | 7.3 |
| PMON70678 | GM_A31341 | 8.7 | 4.0 | 19.5 | 59.4 | 7.8 |
| PMON70678 | GM_A31345 | 8.8 | 5.1 | 15.2 | 62.4 | 8.4 |
| PMON70678 | GM_A31342 | 8.8 | 5.8 | 13.4 | 63.0 | 9.0 |
| PMON70678 | GM_A31345 | 8.9 | 5.2 | 15.3 | 62.0 | 8.7 |
| PMON70678 | GM_A31345 | 8.9 | 5.6 | 15.0 | 61.9 | 8.4 |
| PMON70678 | GM_A31341 | 8.9 | 3.3 | 38.8 | 43.2 | 5.3 |
| PMON70678 | GM_A31345 | 9.0 | 5.1 | 16.6 | 60.7 | 8.5 |
| PMON70678 | GM_A31342 | 9.0 | 5.5 | 16.2 | 61.9 | 7.2 |
| PMON70678 | GM_A31341 | 9.0 | 4.1 | 31.1 | 49.9 | 5.5 |
| PMON70678 | GM_A31349 | 9.1 | 6.0 | 12.7 | 61.9 | 9.7 |
| PMON70678 | GM_A31342 | 9.1 | 5.2 | 15.4 | 62.5 | 7.8 |
| PMON70678 | GM_A31417 | 9.2 | 5.6 | 15.1 | 60.8 | 9.0 |
| PMON70678 | GM_A31349 | 9.2 | 5.5 | 14.0 | 62.2 | 9.2 |
| PMON70678 | GM_A31350 | 9.2 | 4.6 | 18.5 | 58.8 | 8.5 |
| PMON70678 | GM_A31342 | 9.4 | 5.1 | 15.5 | 62.2 | 7.5 |
| PMON70678 | GM_A31350 | 9.5 | 5.3 | 14.7 | 61.5 | 8.6 |
| PMON70678 | GM_A31417 | 9.5 | 5.3 | 15.3 | 60.9 | 8.6 |
| PMON70678 | GM_A31345 | 9.5 | 5.7 | 14.6 | 61.2 | 8.8 |
| PMON70678 | GM_A31350 | 9.6 | 5.5 | 13.7 | 61.7 | 9.1 |
| PMON70678 | GM_A31417 | 9.6 | 5.2 | 16.0 | 60.0 | 8.8 |
| PMON70678 | GM_A31341 | 9.6 | 3.5 | 24.6 | 54.9 | 6.9 |
| PMON70678 | GM_A31341 | 9.7 | 3.7 | 20.7 | 58.5 | 6.7 |
| PMON70678 | GM_A31341 | 9.8 | 3.8 | 19.6 | 58.5 | 7.7 |
| PMON70678 | GM_A31345 | 9.9 | 5.1 | 14.8 | 61.4 | 8.6 |
| control | A3244 | 12.4 | 4.3 | 18.3 | 56.4 | 8.0 |
| control | A3244 | 12.4 | 6.7 | 14.0 | 57.1 | 8.8 |
| control | A3244 | 12.6 | 4.9 | 15.4 | 57.4 | 9.1 |
| control | A3244 | 12.9 | 5.0 | 17.6 | 55.9 | 8.2 |
| control | A3244 | 12.9 | 4.9 | 14.4 | 57.5 | 9.8 |
| control | A3244 | 13.0 | 4.7 | 14.6 | 55.6 | 9.7 |
| control | A3244 | 13.0 | 4.7 | 14.9 | 57.0 | 9.4 |
| control | A3244 | 13.0 | 5.0 | 13.8 | 57.4 | 10.2 |
| control | A3244 | 13.2 | 4.5 | 16.9 | 54.6 | 7.8 |
| control | A3244 | 13.3 | 5.1 | 14.1 | 57.8 | 9.4 |

### Example 4

RNA is isolated from homozygous R₂ seed from two FATB intron suppressed lines, and from negative controls (wild type seed and seed from null segregants from each intron suppressed event). Northern gels containing these RNA samples are probed with the FATB cDNA. FATB transcript levels are significantly reduced in the intron suppressed lines relative to the negative controls.

### Example 5

### FATB Intron Constructs

Plant expression constructs are made containing one or more FATB introns in sense or antisense orientation. To achieve a desired fatty acid effect, two or more FatB introns are combined into one transcriptional unit. In an alternate approach, each FATB intron is expressed under the contol of its own promoter (monocistronic). Other constructs are made where a FATB intron is capable of producing dsRNA, either using only one transcriptional unit (inverted repeat) or two expression cassettes, with one containing a sense intron and the other containing an antisense intron.

These constructs are stably introduced into soybean (for example, Asgrow variety A3244) by the methods described earlier. Transformed soybean plants are identified by selection on media containing glyphosate. Gas chromatography is used to determine the fatty acid composition of seed from soybean lines transformed with the constructs. In addition, any of the constructs can contain other sequences of interest, including without limitation, sequences to overexpress KAS I, KAS IV, and/or delta-9 desaturase, as well as different combinations of promoters.

1. A recombinant nucleic acid molecule comprising as operably linked components: (A) a promoter that functions in a plant cell to cause production of an mRNA molecule; and (B) a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of at least 15 contiguous nucleotides of either.

2. The recombinant nucleic acid molecule of claim 1, wherein the promoter is a seed-specific promoter.

3. The recombinant nucleic acid molecule of claim 2, wherein the promoter is a 7S promoter.

4. The recombinant nucleic acid molecule of claim 1, wherein the nucleic acid sequence is in a sense orientation relative to the promoter.

5. The recombinant nucleic acid molecule of claim 1, wherein the nucleic acid sequence is in an antisense orientation relative to the promoter.

6. The recombinant nucleic acid molecule of claim 1, wherein the nucleic acid sequence is capable of expressing a dsRNA.

7. The recombinant nucleic acid molecule of claim 1, wherein said nucleic acid molecule further comprises one or more additional nucleic acid sequences, wherein said additional nucleic acid sequences encode an enzyme selected from the group consisting of beta-ketoacyl-ACP synthase I, beta-ketoacyl-ACP synthase IV, and delta-9 desaturase.

8. The recombinant nucleic acid molecule of claim 7, wherein the additional nucleic acid sequence encodes beta-ketoacyl-ACP synthase IV.

9. The recombinant nucleic acid molecule of claim 7, wherein the additional nucleic acid sequences encode beta-ketoacyl-ACP synthase IV and delta-9 desaturase.

10. The recombinant nucleic acid molecule of claim 1, wherein said fragments are fragments of at least 25 contiguous nucleotides.

11. The recombinant nucleic acid molecule of claim 1, wherein said fragments are fragments of at least 25 contiguous nucleotides.

12. An isolated polynucleotide sequence selected from the group consisting of:
a) a polynucleotide sequence having at least 70% identity to coding regions of SEQ ID NO: 1 over the entire length of SEQ ID NO: 1 or fragments of at least 15 contiguous nucleotides thereof;
b) a polynucleotide sequence having at least 80% identity to coding regions of SEQ ID NO: 1 over the entire length of SEQ ID NO: 1 or fragments of at least 15 contiguous nucleotides thereof;
c) a polynucleotide sequence having at least 90% identity to coding regions of SEQ ID NO: 1 over the entire length of SEQ ID NO: 1 or fragments of at least 15 contiguous nucleotides thereof; and
d) a polynucleotide sequence having at least 95% identity to coding regions of SEQ ID NO: 1 over the entire length of SEQ ID NO: 1 or fragments of at least 15 contiguous nucleotides thereof.

13. An isolated polynucleotide sequence selected from the group consisting of:
a) a polynucleotide sequence having at least 70% identity to coding regions of SEQ ID NO: 10 over the entire length of SEQ ID NO: 10 or fragments of at least 15 contiguous nucleotides thereof;
b) a polynucleotide sequence having at least 80% identity to coding regions of SEQ ID NO: 10 over the entire length of SEQ ID NO: 10 or fragments of at least 15 contiguous nucleotides thereof;
c) a polynucleotide sequence having at least 90% identity to coding regions of SEQ ID NO: 10 over the entire length of SEQ ID NO: 10 or fragments of at least 15 contiguous nucleotides thereof; and
d) a polynucleotide sequence having at,least 95% identity to coding regions of SEQ ID NO: 10 over the entire length of SEQ ID NO: 10 or fragments of at least 15 contiguous nucleotides thereof.

14. A transformed soybean plant comprising a recombinant nucleic acid molecule, the recombinant nucleic acid molecule comprising as operably linked components: (A) a promoter that functions in a plant to cause production of an mRNA molecule; and (B) a nucleic acid sequence that has at least 85% identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of at least 15 contiguous nucleotides of either.

15. The transformed plant of claim 14, wherein said transformed plant exhibits a reduced palmitic acid level relative to a plant with a similar genetic background but lacking the recombinant nucleic acid molecule.

16. The transformed plant of claim 14, wherein said transformed plant produces a seed with a reduced palmitic acid level relative to a plant with a similar genetic background but lacking the recombinant nucleic acid molecule.

17. The transformed plant of claim 14, wherein said transformed plant exhibits a reduced stearic acid level relative to a plant with a similar genetic background but lacking the recombinant nucleic acid molecule.

18. The transformed plant of claim 14, wherein said transformed plant produces a seed with a reduced stearic acid level relative to a plant with a similar genetic background but lacking the recombinant nucleic acid molecule.

19. The transformed plant of claim 14, wherein said transformed plant produces a seed with a reduced saturated fatty acid content relative to a plant with a similar genetic background but lacking the recombinant nucleic acid molecule.

20. The transformed plant of claim 14, wherein said transformed plant exhibits an increased oleic acid level relative to a plant with a similar genetic background but lacking the recombinant nucleic acid molecule.

21. The transformed plant of claim 14, wherein said transformed plant produces a seed with an increased oleic acid level relative to a plant with a similar genetic background but lacking the recombinant nucleic acid molecule.

22. The transformed plant of claim 14, wherein said fragments are fragments of at least 25 contiguous nucleotides.

23. The transformed plant of claim 14, wherein said fragments are fragments of at least 25 contiguous nucleotides.

24. A transformed soybean plant having a nucleic acid molecule that comprises (a) a first promoter operably linked to a first nucleic acid molecule having a first nucleic acid sequence that has 85% or greater identity to a nucleic acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of at least 15 contiguous nucleotides of either, and (b) a second nucleic acid molecule with a second nucleic acid sequence that encodes an enzyme selected from the group consisting of beta-ketoacyl-ACP synthase I, beta-ketoacyl-ACP synthase IV, and delta-9 desaturase.

25. The transformed soybean plant according to claim 24, wherein the first promoter is a seed specific promoter.

26. The transformed soybean plant according to claim 24, wherein the first promoter is a 7S promoter.

27. The transformed soybean plant according to claim 24, wherein said first nucleic acid molecule is transcribed and is capable of at least partially reducing the level of a transcript encoded by an endogenous FATB gene.

28. The transformed soybean plant of claim 24, wherein said fragments are fragments of at least 25 contiguous nucleotides.

29. The transformed soybean plant of claim 24, wherein said fragments are fragments of at least 25 contiguous nucleotides.

30. A method of modifying the lipid composition in a host cell comprising: providing a host cell with a DNA construct comprising as operably associated components in the 5′ to 3′ direction of transcription, a transcriptional initiation region functional in said host cell, a DNA sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, complements thereof, and fragments of at least 15 contiguous nucleotides of either, and a transcription termination sequence, and growing said cell under conditions wherein transcription of said DNA sequence is initiated, whereby said lipid composition is modified.


SEQUENCE LISTING (20 sequences)

SEQ ID NO: 1 - DNA, 4086 bp, Glycine max
  soybean FATB genomic clone
ttagggaaacaacaaggacgcaaaatgacacaatagcccttcttccctgtttccagcttttctccttctctctctccatcttcttcttcttcttcactcagtcaggtacgcaaacaaatctgctattcattcattcattcctctttctctctgatcgcaaactgcacctctacgctccactcttctcattttctcttcctttctcgcttctcagatccaactcctcagataacacaagaccaaacccgctttttctgcatttctagactagacgttctaccggagaaggttctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaaggtgggagggaaaatgtattttttttttctcttctaacttgcgtatattttgcatgcagcgaccttagaaattcattatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaggcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagtaagtccgtcctcatacaagtgaatctttatgatcttcagagatgagtatgctttgactaagatagggctgtttatttagacactgtaattcaatttcatatatagataatatcattctgttgttacttttcatactatatttatatcaactatttgcttaacaacaggaaactgcacttaatcatgttaaaagtgctgggcttcttggtgatggctttggttccacgccagaaatgtgcaaaaagaacttgatatgggtggttactcggatgcaggttgtggtggaacgctatcctacatggttagtcatctagattcaaccattacatgtgatttgcaatgtatccatgttaagctgctatttctctgtctattttagtaatctttatgaggaatgatcactcctaaatatattcatggtaattattgagacttaattatgagaaccaaaatgctttggaaatttgtctgggatgaaaattgattagatacacaagctttatacatgatgaactatgggaaaccttgtgcaacagagctattgatctgtacaagagatgtagtatagcattaattacatgttattagataaggtgacttatccttgtttaattattgtaaaaatagaagctgatactatgtattctttgcatttgttttcttaccagttatatataccctctgttctgtttgagtactactagatgtataaagaatgcaattattctgacttcttggtgttgggttgaagttagataagctattagtattattatggttattctaaatctaattatctgaaattgtgtgtctatatttgcttcaggggtgacatagttcaagtggacacttgggtttctggatcagggaagaatggtatgcgtcgtgattggcttttacgtgactgcaaaactggtgaaatcttgacaagagcttccaggtagaaatcattctctgtaattttccttcccctttccttctgcttcaagcaaattttaagatgtgtatcttaatgtgcacgatgctgattggacacaattttaaatctttcaaacatttacaaaagttatggaaccctttcttttctctcttgaagatgcaaatttgtcacgactgaagtttgaggaaatcatttgaattttgcaatgttaaaaaagataatgaactacatattttgcaggcaaaaacctctaattgaacaaactgaacattgtatcttagtttatttatcagactttatcatgtgtactgatgcatcaccttggagcttgtaatgaattacatattagcattttctgaactgtatgttatggttttggtgatctacagtgtttgggtcatgatgaataagctgacacggaggctgtctaaaattccagaagaagtcagacaggagataggatcttattttgtggattctgatccaattctagaagaggataacagaaaactgactaaacttgacgacaacacagcggattatattcgtaccggtttaagtgtatgtcaactagtttttttgtaattgttgtcattaatttcttttcttaaattatttcagatgttgctttctaattagtttacattatgtatcttcattcttccagtctaggtggagtgatctagatatcaatcagcatgtcaacaatgtgaagtacattgactggattctggaggtatttttctgttcttgtattctaatccactgcagtccttgttttgttgttaaccaaaggactgtcctttgattgtttgcagagtgctccacagccaatcttggagagtcatgagctttcttccgtgactttagagtataggagggagtgtggtagggacagtgtgctggattccctgactgctgtatctggggccgacatgggcaatctagctcacagtggacatgttgagtgcaagcatttgcttcgactcgaaaatggtgctgagattgtgaggggcaggactgagtggaggcccaaacctatgaacaacattggtgttgtgaaccaggttccagcagaaagcacctaagattttgaaatggttaacggttggagttgcatcagtctccttgctatgtttagacttattctggcctctggggagagttttgcttgtgtctgtccaatcaatctacatatctttatatccttctaatttgtgttactttggtgggtaagggggaaaagctgcagtaaacctcattctctctttctgctgctccatatttcatttcatctctgattgcgctactgctaggctgtcttcaatatttaattgcttgatcaaaatagctaggcatgtatattattattcttttctcttggctcaattaaagatgcaattttcattgtgaacacagcataactattattcttattatttttgtatagcctgtatgcacgaatgacttgtccatccaatacaaccgtgattgtatgctccagctcag

SEQ ID NO: 2 - DNA, 104 bp, Glycine max
  soybean FATB intron I
caaatctgctattcattcattcattcctctttctctctgatcgcaaactgcacctctacgctccactcttctcattttctcttcctttctcgcttctcagatcc

SEQ ID NO: 3 - DNA, 839 bp, Glycine max
  soybean FATB intron II
ctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaggtgggagggaaaatgtattttttttttctcttctaacttgcgtatattttgcatgcagcgacct

SEQ ID NO: 4 - DNA, 169 bp, Glycine max
  soybean FATB intron III
taagtccgtcctcatacaagtgaatctttatgatcttcagagatgagtatgctttgactaagatagggctgtttatttagacactgtaattcaatttcatatatagataatatcattctgttgttacttttcatactatatttatatcaactatttgcttaacaacagg

SEQ ID NO: 5 - DNA, 525 bp, Glycine max
  FATB intron IV
gttagtcatctagattcaaccattacatgtgatttgcaatgtatccatgttaagctgctatttctctgtctattttagtaatctttatgaggaatgatcactcctaaatatattcatggtaattattgagacttaattatgagaaccaaaatgctttggaaatttgtctgggatgaaaattgattagatacacaagctttatacatgatgaactatgggaaaccttgtgcaacagagctattgatctgtacaagagatgtagtatagcattaattacatgttattagataaggtgacttatccttgtttaattattgtaaaaatagaagctgatactatgtattctttgcatttgttttcttaccagttatatataccctctgttctgtttgagtactactagatgtataaagaatgcaattattctgacttcttggtgttgggttgaagttagataagctattagtattattatggttattctaaatctaattatctgaaattgtgtgtctatatttgcttcag

SEQ ID NO: 6 - DNA, 389 bp, Glycine max
  FATB intron V
gtagaaatcattctctgtaattttccttcccctttccttctgcttcaagcaaattttaagatgtgtatcttaatgtgcacgatgctgattggacacaattttaaatctttcaaacatttacaaaagttatggaaccctttcttttctctcttgaagatgcaaatttgtcacgactgaagtttgaggaaatcatttgaattttgcaatgttaaaaaagataatgaactacatattttgcaggcaaaaacctctaattgaacaaactgaacattgtatcttagtttatttatcagactttatcatgtgtactgatgcatcaccttggagcttgtaatgaattacatattagcattttctgaactgtatgttatggttttggtgatctacag

SEQ ID NO: 7 - DNA, 106 bp, Glycine max
  FATB intron VI
tatgtcaactagtttttttgtaattgttgtcattaatttcttttcttaaattatttcagatgttgctttctaattagtttacattatgtatcttcattcttccagt

SEQ ID NO: 8 - DNA, 82 bp, Glycine max
  FATB intron VII
gtatttttctgttcttgtattctaatccactgcagtccttgttttgttgttaaccaaaggactgtcctttgattgtttgcag

SEQ ID NO: 9 - protein, 328 aa, Glycine max
  soybean FATB enzyme
MEEQLLAAITTIFLAAEKQWMMLDWKPRRPDMLIDPFGIGKIVQDGLVFRENFSIRSYEIGADRTASIETVMNHLQETALNHVKSAGLLGDGFGSTPEMCKKNLIWVVTRMQVVVERYPTWGDIVQVDTWVSGSGKNGMRRDWLLRDSKTGEILTRASSVWVMMNKLTRRLSKIPEEVRQEIGSYFVDSDPILEEDNRKLTKLDDNTADYIRTGLSPRWSDLDINQHVNNVKYIGWILESAPQPILESHELSSMTLEYRRECGRDSVLDSLTAVSGADMGNLAHSGHVECKHLLRLENGAEIVRGRTEWRPKPVNNFGVVNQVPAEST

SEQ ID NO: 10 - DNA, 1856 bp, Glycine max
  soybean FATB partial genomic clone
ttagggaaacaacaaggacgcaaaatgacacaatagcccttcttccctgtttccagcttttctccttctctctctccatcttcttcttcttcttcactcagtcaggtacgcaaacaaatctgctattcattcattcattcctctttctctctgatcgcaaactgcacctctacgctccactcttctcattttctcttcctttctcgcttctcagatccaactcctcagataacacaagaccaaacccgctttttctgcatttctagactagacgttctaccggagaaggttctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaggtgggagggaaaatgtattttttttttctcttctaacttgcgtatattttgcatgcagcgaccttagaaattcattatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaggcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagtaagtccgtcctcatacaagtgaatctttatgatcttcagagatgagtatgctttgactaagatagggctgtttatttagacactgtaattcaatttcatatatagataatatcattctgttgttacttttcatactatatttatatcaactatttgcttaacaacaggaaactgcacttaatcatgttaaaagtgctgggcttcttggtgatggctggta

SEQ ID NO: 11 - DNA, 34 bp, Artificial
  Oligonucleotide primer F1
gcggccgccccgggttagggaaacaacaaggacg

SEQ ID NO: 12 - DNA, 34 bp, Artificial
  Oligonucleotide primer F2
gcggccgccccgggcagtcagatccaactcctca

SEQ ID NO: 13 - DNA, 34 bp, Artificial
  Oligonucleotide primer F3
gcggccgccccgggattggtgctgatcgtaccgc

SEQ ID NO: 14 - DNA, 38 bp, Artificial
  Oligonucleotide primer R1
gcggccgcggtacccccccttacccaccaaagtatcac

SEQ ID NO: 15 - DNA, 34 bp, Artificial
  Oligonucleotide primer R2
gcggccgcggtaccaaactctccccagggaacca

SEQ ID NO: 16 - DNA, 34 bp, Artificial
  Oligonucleotide primer R3
gcggccgcggtaccagccatcaccaagaagccca

SEQ ID NO: 17 - DNA, 37 bp, Artificial
  Oligonucleotide primer 18133
gaattcctcgagctcgattcttttctcttttaacttt

SEQ ID NO: 18 - DNA, 37 bp, Artificial
  Oligonucleotide primer 18134
gaattcctcgagcatgcaaaatatacgcaagttagaa

SEQ ID NO: 19 - DNA, 854 bp, Artificial
  PCR product containing soybean FATB intron II
gaattcctcgagctcgattcttttctcttttaactttatttttaaaataataataatgagagctggatgcgtctgttcgttgtgaatttcgaggcaatggggttctcattttcgttacagttacagattgcattgtctgctttcctcttctcccttgtttctttgccttgtctgatttttcgtttttatttcttacttttaatttttggggatggatattttttctgcattttttcggtttgcgatgttttcaggattccgattccgagtcagatctgcgccggcttatacgacgaatttgttcttattcgcaacttttcgcttgattggcttgttttacctctggaatctcacacgtgatcaaataagcctgctattttagttgaagtagaatttgttctttatcggaaagaattctatggatctgttctgaaattggagctactgtttcgagttgctattttttttagtagtattaagaacaagtttgccttttattttacatttttttcctttgcttttgccaaaagtttttatgatcactctcttctgtttgtgatataactgatgtgctgtgctgttattatttgttatttggggtgaagtataattttttgggtgaacttggagcatttttagtccgattgatttctcgatatcatttaaggctaaggttgacctctaccacgcgtttgcgtttgatgttttttccattttttttttatctcatatcttttacagtgtttgcctatttgcatttctcttctttatcccctttctgtggaaggtgggagggaaaatgtattttttttttctcttctaacttgcgtatattttgcatgctcgaggaattc

SEQ ID NO: 20 - DNA, 1688 bp, Glycine max
  soybean FATB cDNA
acaattacactgtctctctcttttccaaaattagggaaacaacaaggacgcaaaatgacacaatagcccttcttccctgtttccagcttttctccttctctctctctccatcttcttcttcttcttcactcagtcagatccaactcctcagataacacaagaccaaacccgctttttctgcatttctagactagacgttctaccggagaagcgaccttagaaattcattatggtggcaacagctgctacttcatcatttttccctgttacttcaccctcgccggactctggtggagcaggcagcaaacttggtggtgggcctgcaaaccttggaggactaaaatccaaatctgcgtcttctggtggcttgaaggcaaaggcgcaagccccttcgaaaattaatggaaccacagttgttacatctaaagaaagcttcaagcatgatgatgatctaccttcgcctccccccagaacttttatcaaccagttgcctgattggagcatgcttcttgctgctatcacaacaattttcttggccgctgaaaagcagtggatgatgcttgattggaagccacggcgacctgacatgcttattgacccctttgggataggaaaaattgttcaggatggtcttgtgttccgtgaaaacttttctattagatcatatgagattggtgctgatcgtaccgcatctatagaaacagtaatgaaccatttgcaagaaactgcacttaatcatgttaaaagtgctgggcttcttggtgatggctttggttccacgccagaaatgtgcaaaaagaacttgatatgggtggttactcggatgcaggttgtggtggaacgctatcctacatggggtgacatagttcaagtggacacttgggtttctggatcagggaagaatggtatgcgtcgtgattggcttttacgtgactccaaaactggtgaaatcttgacaagagcttccagtgtttgggtcatgatgaataagctaacacggaggctgtctaaaattccagaagaagtcagacaggagataggatcttattttgtggattctgatccaattctggaagaggataacagaaaactgactaaacttgacgacaacacagcggattatattcgtaccggtttaagtcctaggtggagtgatctagatatcaatcagcatgtcaacaatgtgaagtacattggctggattctggagagtgctccacagccaatcttggagagtcatgagctttcttccatgactttagagtataggagagagtgtggtagggacagtgtgctggattccctgactgctgtatctggggccgacatgggcaatctagctcacagcgggcatgttgagtgcaagcatttgcttcgactggaaaatggtgctgagattgtgaggggcaggactgagtggaggcccaaacctgtgaacaactttggtgttgtgaaccaggttccagcagaaagcacctaagatttgaaatggttaacgattggagttgcatcagtctccttgctatgtttagacttattctggttccctggggagagttttgcttgtgtctatccaatcaatctacatgtctttaaatatatacaccttctaatttgtgatactttggtgggtaagggggaaaagcagcagtaaatctcattctcattgtaattaaaaaaaaaaaaaa